(19) United States
(12) Patent Application Publication
(10) Pub. No.: US 2015/0376219 A1
(43) Pub. Date: Dec. 31, 2015

(54) Selective Preparations of Purine Nucleosides and Nucleotides: Reagents and Methods

(71) Applicant: Minghong Zhong, Altamont, NY (US)

(72) Inventor: Minghong Zhong, Altamont, NY (US)

(21) Appl. No.: 14/748,237

(22) Filed: Jun. 24, 2015

Related U.S. Application Data
(60) Provisional application No. 62/018,696, filed on Jun. 30, 2014.

(51) Int. Cl. C07H 1/00; C07H 19/20

(52) U.S. Cl. CPC C07H 1/00; C07H 19/20

(57) ABSTRACT

A process of regiospecific synthesis of N-9 purine nucleoside analogs in either solution or solid phase synthesis is described. The introduction of the sugar moiety or its analogue on to a 6-heteroarylium purine or its mesomeric betaine so that formation of only the N-9 position regioisomers of the purine nucleoside analogs (either D or L enantiomers) is obtained. This regiospecific introduction of the sugar moiety allows the synthesis of purine nucleoside analogs in high yields without formation of the N-7-positional regioisomers, while the 6-heteroaryliums are leaving groups facilitated for nucleophilic displacement. Solid supported 6-heterarylium purine bases can be used for purine based library synthesis and synthesis of nucleotide monophosphates and polyphosphates. Processes for providing novel 6-heteroarylium purines and their corresponding mesomeric betaines for the regiospecific synthesis of N-9 purine nucleoside analogs and nucleotides are described.

### CROSS REFERENCE TO RELATED APPLICATIONS

The present applications claims the benefits of U.S. Provisional Application Ser. No. 62/018,696, the entire said invention being incorporated herein by reference.

### TECHNICAL FIELD OF THE INVENTION

The present invention relates to substrates and methods for producing purine nucleosides through N-9 regiospecific glycosylation or alkylation of 6-heteroarylium purines and their mesomeric betaines, and facilitated displacements at C-6 of the resulting glycosylated or alkylated purines, and for preparation of nucleoside ProTides, and mono-, di-, triphosphates through synthesis on solid supports tethered at 6-heteroarylium moieties of corresponding nucleosides, followed by the aforementioned facilitated displacements.

### BACKGROUND OF THE INVENTION

Nucleoside analogues are highly effective agents for treatment of viral infectious diseases such as AIDS, hepatitis B, hepatitis C, herpes virus, herpes zoster, cytomegalovirus and the like, and also effective for treatment of neoplastic diseases.

Examples of therapeutic nucleoside analogues include Entecavir, Abacavir, Tenofovir, Adefovir, Acyclovir, Ganciclovir, Famciclovir, Lobucavir, Cladribine (2-CdA), Fludarabine, Clofarabine, Nelarabine, Gemcitabine, Lamivudine, Telbivudine, Sovaldi, and others. Notably, the therapeutic purine derivatives have a sugar substituent or its equivalents at the 9-position, not the 7-position.

Purine nucleoside can be prepared by coupling a purine with sugar derivative or its analogue via glycosylation or alkylation (convergent synthesis), by construction of the purine core from a primary amine (linear synthesis), or by derivatizations of a natural purine nucleoside (adenosine, guanosine, and inosine) and 2′-deoxy purine nucleoside (2′-deoxyadenosine and 2′-deoxyguanosine). A convergent synthesis is a method of choice, and provides shorter and more efficient routes with better overall yields, and more diverse structures in products. However, achieving regiospecific alkylation and glycosylation of purine derivatives at the 9-position is challenging. 6-Chloropurines such as 6-chloropurine, 2,6-dichloropurine, 2-amino-6-chloropurine, and 2-amino protected 2-amino-6-chloropurine are widely used key intermediates for manufacturing of many purine drugs, because of their easy access to many diversified structures by further exclusive displacement of 6-chloro with nucleophiles such as ammonia, amine, hydroxide, alkoxide, hydrogen, and others; glycosylation and alkylation of 6-chloropurines under various conditions usually give a mixture of N9-/N7-isomers. Alkylation and glycosylation of guanine, hypoxanthine, 6-alkoxyguanine and 6-alkoxypurine, or their persilylated forms, gave a mixture of N9-/N7-isomers universally. Selectivity in alkylation and glycosylation of adenine, 2-chloroadenine, 6-acylated or 6-benzylated adenine, or their persilylated forms varies by cases, and many examples of alkylation at N7, 6-amino, or both, as side reactions were reported.

The complicacy in convergent synthesis is further increased by problematic stereoselectivity for 2-deoxysugars and other sugars (such as arabinose) that lack correct neighboring group participation. Glycosylation procedures in which a 2-deoxysugar moiety is coupled with an aglycon invariably provide positional isomers (regio-isomers) as well as anomeric mixtures, which can result in low yields of the desired nucleoside and often requires troublesome purification protocols.

A simplified procedure for N-9 glycosylation that is regiospecific would be highly desirable. Numerous efforts to enhance N-9 regioselective glycosylation have been made, as reviewed in literature (Robins and Zhong, WO 2006138396). An effective method was recently reported by Zhong and Robins (J. Org. Chem. 2006, 7773), using 6-(imidazol-1-yl)purines to obtain N-9 glycosylated products exclusively, but this imidazole moiety was difficult to be cleaved by aminolysis. This problem was partially solved by activation of this azole as imidazolium salts by introducing genotoxic benzyl halides and extra steps or under more strenuous reaction conditions, while conversion of 6-imidazolyl to 6-oxo requires multistep chemical transformations.

This regioselectivity issue recurs in 3-deazapurine analogues, and reported efforts include protection of 6-NH₂ with 3,3,4,4-tetramethylsuccimide (TMSI) with the selectivity problem still existent and its synthetic application is limited to adenine-like nucleosides (McLaughlin, et al., Org. Lett. 2010, 120).

Another challenge in discovery of therapeutic nucleosides is synthesis and purification of nucleotides, especially nucleoside polyphosphates.

Therapeutic nucleosides are usually required of sequential phosphorylations to be activated as inhibitors or substrates of targeted enzymes. Production of nucleotide 5′-phosphates and 5′-polyphosphates has problems of low reaction selectivity and difficult purifications, which hurdles the drug discovery and development based on these compound libraries and on enzymatic synthesis of long RNAs using nucleotide triphosphates. Solid supported synthesis has advantages in removal of excess of phosphorylating reagents such as pyrophosphate salts by simple washings, and convenient access to nucleotide libraries. But compatible linker chemistry to labile triphosphates is required for such an application, in addition to the requirement of protection of both base and sugar moieties. Reported examples of solid phase synthesis of nucleoside triphosphates include synthesis based on solid supported mono-, di-, and triphosphitylating reagents (Parang, et al. Curr. Protoc. Nucleic Acid Chem, 2008), which is not practical and inapplicable to compound library synthesis because of its needs of expensive nucleosides of usually low solubility in great excess, and soluble poly(ethylene glycol) supported synthesis of cytosine-containing nucleosides (Peyrottes, et al. J. Org. Chem. 2011).

This invention introduces 6-heteroarylium purines for N-9 regiospecific alkylation and glycosylation, with neutral heteroarenes as much better leaving groups than anionic imidazolides (pKa˜18.6). Solid phase tethered nucleosides at 6-heteroaryliums of this invention in combination of compatible protection groups can facilitate cost-effective productions of nucleotides.

### SUMMARY OF THE INVENTION

The invention provides versatile reagents and methods for regiospecific synthesis of N9 purine nucleosides including purine ribonucleoside analogs, 2′-deoxy, 3′-deoxy, 2′-deoxy-2′-halo-arabino, 2′,3′-dideoxy-2′-halo-threo purine nucleoside analogs, acyclic, and carbocyclic purine nucleosides, without formation of the 7-positional regioisomers. These reagents include a 6-heteroarylium such as 6-(4-N,N-dialkylaminopyridin-1-ium-1-yl) (pKa=9.2), 6-(3-N-alkylimidazol-1-ium-1-yl) (pKa=7.0), 6-(4-alkoxylpyridin-1-ium-1-yl) (pKa=6.6), or 6-(4-alkylthiopyridin-1-ium-1-yl) (pKa˜5-6)), or 6-(pyridin-1-ium-1-yl) (pKa˜5.2) as N9-directing auxiliaries and facilitated good leaving groups, are zwitterionic when deprotonated, and have a locked coplanar conformation.

When applied to solid supported synthesis, in combination with compatible protections, libraries of purine nucleosides can be synthesized, and the derived solid supported nucleoside products can be phosphorylated to provide libraries of nucleoside ProTides and phosphates after global deprotections/cleavage of the solid supports.

In one embodiment, the invention provides a method that includes (a) glycosylating or alkylating a 6-heteroaryliumpurin-9-ide at the N-9 position either on solid supports or in solution and (b) displacing the 6-heteroarylium group of the 6-heteroarylium purine nucleoside from step (a) with a nucleophile to yield an N-9 purine nucleoside.

In one embodiment, the invention provides a method that includes (a) glycosylating a 6-(4-N,N-dialkylaminopyridin-1-ium-1-yl)purin-9-ide at the N-9 position either on solid supports or in solution and (b) displacing the 6-(pyridin-1-ium-1-yl) group from the glycosylated purine from step (a) with a nucleophile to yield an N-9 purine nucleoside.

In one embodiment, the invention provides a method that includes (a) glycosylating a 6-(3-N-alkylimidazol-1-ium-1-yl)purin-9-ide at the N-9 position either on solid supports or in solution and (b) displacing the 6-(imidazol-1-ium-1-yl) group from the glycosylated purine from step (a) with a nucleophile to yield an N-9 purine nucleoside.

In one embodiment, the invention provides a method that includes (a) glycosylating a 6-(4-alkoxylpyridin-1-ium-1-yl)purin-9-ide at the N-9 position either on solid supports or in solution and (b) displacing 6-(pyridin-1-ium-1-yl) group from the glycosylated purine from step (a) with a nucleophile to yield an N-9 purine nucleoside.

In one embodiment, the invention provides a method that includes (a) glycosylating a 6-(4-alkylthiopyridin-1-ium-1-yl)purin-9-ide at the N-9 position either on solid supports or in solution and (b) displacing 6-(pyridin-1-ium-1-yl) group from the glycosylated purine from step (a) with a nucleophile to yield an N-9 purine nucleoside.

In another embodiment, the invention provides a method that includes (a) introducing a pyridin-1-ium-1-yl group or an imidazol-1-ium-1-yl at the 6-position of a purine, (b) glycosylating the purine product from step (a) at the N-9 position and (c) displacing the 6-(pyridin-1-ium-1-yl) or 6-(imidazol-1-ium-1-yl) group from step (b) with a nucleophile to yield an N-9 purine nucleoside.

In one embodiment, the invention provides a method that includes (a) glycosylating or alkylating a solid supported a 6-heteroarylium purine or its mesomeric betaine at the N-9 position, (b) deprotection of 5′-OH or global deprotection of sugar moiety of the nucleoside formed in step (a), (c) selective 5′-phosphorylation of the resulting tethered nucleosides from step (b), and (d) displacing the 6-leaving group as a neutral heteroarene such as pyridine or imidazole from the nucleotide from step (c) with a nucleophile, and global deprotection to yield an N-9 purine nucleoside 5′-ProTide, mono-, di-, tri-, or polyphosphates.

In one embodiment, the invention provides a method that includes (a) selective 5′-phosphorylation of a solid supported purine nucleoside tethered at 6-heteroarylium substituents, (b) displacing the 6-leaving group as a neutral heteroarene such as pyridine or imidazole from the nucleotide from step (a) with a nucleophile and global deprotection to yield an N-9 purine nucleoside 5′-ProTide, mono-, di-, tri-, or polyphosphates.

The invention provides a method that includes

(a) contacting a substituted purine of Formula I

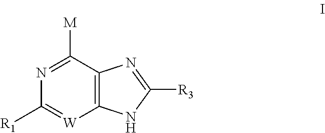

with a glycosylating agent R₆-Lg (Lg is a leaving group) in the presence of a base or a Lewis acid, or an alkylating agent in the presence of a base or a catalyst such as Pd(PPh₃)₄, or under Mitsunobu reaction condition, where W is CH, N, CR₂, and M is a substituted pyridinium, a substituted imidazolium, or 4-oxopyridin-1-yl:

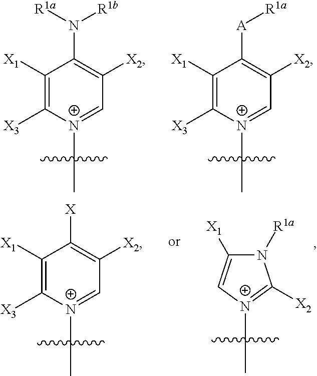

where R₁, R₂, R₃, R^{1a}, R^{1b}, X, X₁, X₂, and X₃ are selected as follows: (i) each R₁, R₂, R₃, X, X₁, X₂, and X₃ is independently selected from hydrogen, C₁₋₁₀ alkyl, C₁₋₁₀ alkoxy, C₁₋₁₀ alkylthio, halogen, amino, hydroxyl, C₁₋₁₀ alkylamino, di-C₁₋₁₀ alkylamino, C₁₋₁₀ acylamino, trialkylsilyl, aryl, and heteroaryl, preferably with X₃ selected as hydrogen; (ii) R^{1a} and R^{1b} is independently selected from C₁₋₁₀ alkyl, aryl, and heteroaryl; (iii) R^{1a}, R^{1b}, X₁, X₂, and X₃ form cyclic rings represented by, but not limited to, the following structures:

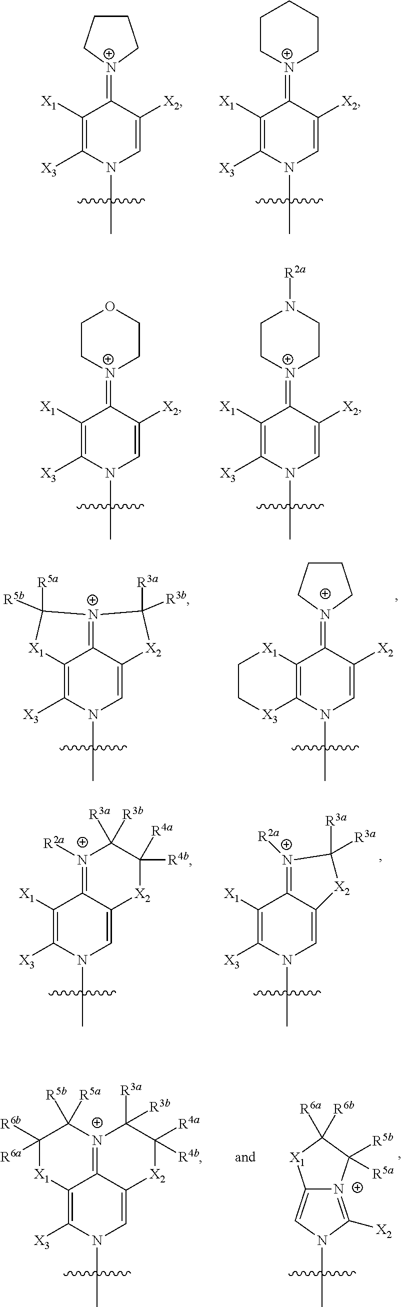

where R^{2a-6a}, and R^{3b-6b} are independently selected from hydrogen, C₁₋₁₀ alkyl, C₁₋₁₀ alkoxy, C₁₋₁₀ alkylthio, halogen, amino, C₁₋₁₀ alkylamino, di-C₁₋₁₀ alkylamino, C₁₋₁₀ acylamino, trialkylsilyl, aryl, and heteroaryl, preferably with X₃ selected as hydrogen; (iv) R^{1a}, R^{1b}, X, X₁, X₂ or X₃ is bound to an inorganic and/or organic particulate solid support as represented by non-limiting examples:

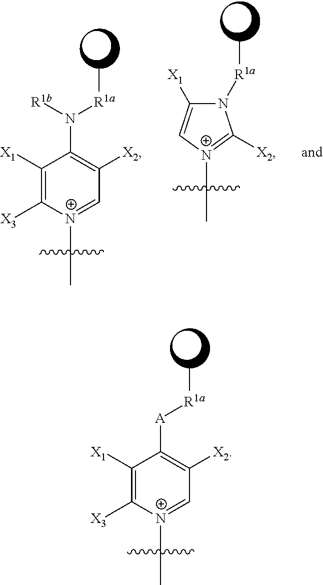

Non-limiting examples of these solid supports include silica gel, silicates, alumina, glass beads, polystyrenes, polyacrylates, and other organic resins; where A is O, S, or alkyl.

(b) contacting the 6-substituted purine nucleoside from step (a) with a nucleophile to obtain a nucleoside of Formula II

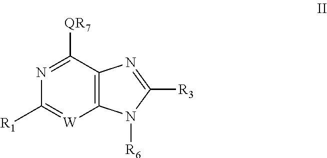

where R₆ is a glycosyl or alkyl group, and Q is independently selected from O, NH, S, and Se, and where R₇ is hydrogen, alkyl, aryl, or heteroaryl.

In some embodiments, a method of the invention involves a 6-substituted purine of Formula III

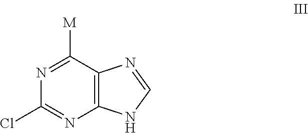

where M is defined as above.

In some embodiments, a method of the invention involves a 6-substituted purine of Formula IV.

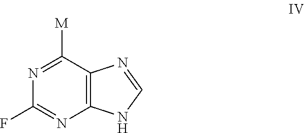

In some embodiments, a method of the invention involves a 6-substituted purine of Formula V where R₄ is acyl or other amine protecting groups.

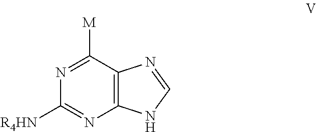

In some embodiments, a method of preparing 2-chloro-2′-deoxyadenosine (2-CdA, Cladribine) comprises (a) contacting a compound having Formula III

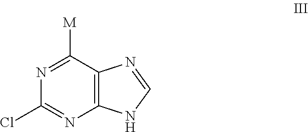

with a base in a first polar solvent followed by contacting an activated and hydroxyl-protected 2-deoxy-α-D-erythro-pentofuranosyl compound in a second less polar solvent to form a glycosylated product, (b) contacting the glycosylated product from step (a) with ammonia in a third solvent followed by deprotection as needed to obtain Cladribine.

In some embodiments, a method of preparing 6-amino-2-fluoroarabino furanosylpurine (Fludarabine) comprises (a) contacting a compound having Formula IV

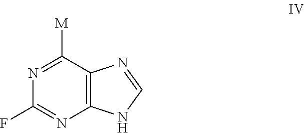

with a base in a first polar solvent followed by contacting an activated and hydroxyl-protected arabinofuranosyl compound in a second less polar solvent to form a glycosylated product, (b) contacting the glycosylated product from step (a) with ammonia in a third solvent followed by deprotection as needed to obtain Fludarabine.

In some embodiments, a method for preparing 2-chloro-2′-β-fluoro-2′-deoxyadenosine (Clofarabine) comprises (a) contacting a compound having Formula III

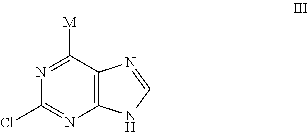

with a base in a first polar solvent followed by contacting an activated and hydroxyl-protected 2-fluoro-2-deoxy-α-D-arabinofuranosyl compound in a second less polar solvent to form a glycosylated product, (b) contacting the 6-substituted purine nucleoside from step (a) with ammonia in a third solvent followed by deprotection as needed to obtain Clofarabine.

In some embodiments, a method of preparing Abacavir, Entecavir, Nelarabine, Lobucavir, Acyclovir, Valacyclovir, Ganciclovir, and Valganciclovir involves a compound having Formula V.

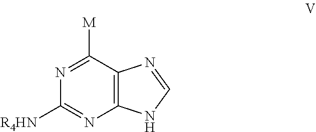

In some embodiments, a method of preparing Didanosine (DDI), Tenofovir and Adefovir involves a compound having Formula VI.

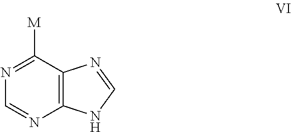

In some embodiments, a method for preparing a purine and a deazapurine of Formula I

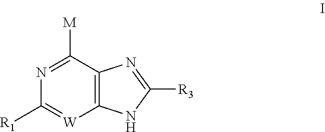

where W is selected from —N—, —CH— and CR₂, includes contacting a compound of Formula VII

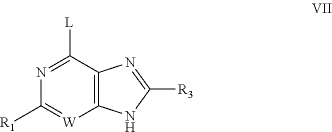

, where L is a halogen, or sulfonate, or other leaving groups, with a pyridine or an imidazole.

In some embodiments, a method for preparing 2-fluoropurine of Formula IV

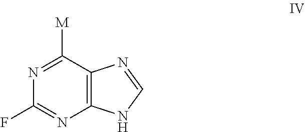

, includes contacting a compound of Formula V

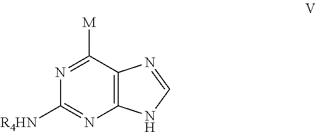

, where R₄ is a hydrogen, with a diazotizing reagent and a fluorinating reagent, sequentially.

In some embodiments of the invention, a compound of Formula I

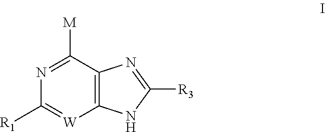

is described where W is selected from —N—, —CH— and CR₂, and M is a substituted pyridinium or a substituted imidazolium

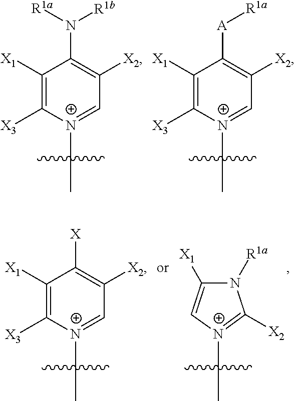

where R₁, R₂, R₃, R^{1a}, R^{1b}, X, X₁, X₂, and X₃ are selected as follows: (i) each R₁, R₂, R₃, X, X₁, X₂, and X₃ is independently selected from hydrogen, C₁₋₁₀ alkyl, C₁₋₁₀ alkoxy, C₁₋₁₀ alkylthio, halogen, amino, C₁₋₁₀ alkylamino, di-C₁₋₁₀ alkylamino, C₁₋₁₀ acylamino, trialkylsilyl, aryl, and heteroaryl, preferably with X₃ selected as hydrogen; (ii) R^{1a} and R^{1b} is independently selected from C₁₋₁₀ alkyl, aryl, and heteroaryl; (iii) R^{1a}R^{1b}, X₁, and X₂ form cyclic rings as described above; (iv) R^{1a}, R^{1b}, X, X₁, X₂ or X₃ is bound to an inorganic and/or organic particulate solid support; and where A is O, S, or alkyl; and pharmaceutically acceptable salts of these compounds.

### BRIEF DESCRIPTION OF THE DRAWINGS

FIG. 1 shows exemplary locked coplanar conformations preferred by effective N9-directing reagents.

FIG. 2 shows examples of solid supported mesomeric purine betaines as effective N9-directing reagents.

FIG. 3 shows examples of resonant structures of 6-(4-N,N-dimethylaminopyridin-1-ium-1-yl)purine and 6-(4-N,N-dimethylaminopyridin-1-ium-1-yl)purin-9-ide.

FIG. 4 shows examples of nucleoside drug compounds and investigational drug compounds manufactured by N-9 glycosylation or N-9 alkylation.

### DETAILED DESCRIPTION OF THE INVENTION

The term “alkyl” as used herein means aliphatic carbon substituents of the alkane, alkene and alkyne families, straight-chain or branched-chain, with or without other substituents on the carbon atoms of the chain, and also includes cyclic-“alkyl” substituents of the noted categories.

The term “aglycon” as used herein means the non-sugar component of a glycoside molecule that results from hydrolysis of the molecule.

The term “glycosyl group” as used herein means the structure obtained by removing the hydroxyl group from the hemiacetal function of a protected or unprotected monosaccharide or a lower oligosaccharide.

The term “glycoside” as used herein means the attachment of a glycosyl group to a non-acyl group, particularly N-glycosides. The bond between the glycosyl group and the non-acyl group is called a glycosidic or glycosyl bond.

The term “nucleoside” as used herein refers to a molecule composed of a heterocyclic nitrogenous base, particularly a purine, containing an N-glycosidic linkage with a sugar, particularly a pentose. Nucleosides include both L- and D-nucleoside enantiomers. Only the structures of the D enantiomers are shown in all drawings; the enantiomeric L structures are the mirror images of the D isomers shown. An extended term of “nucleoside” as used herein also refers to acyclic nucleosides and carbocyclic nucleosides.

The term “ribofuranosyl nucleoside” as used herein refers to a nucleoside or nucleoside analog containing a 2′-hydroxyl group in an L- or D-β-ribofuranosyl configuration.

The term “arabinofuranosyl nucleoside” as used herein refers to a nucleoside or nucleoside analog containing a 2′-hydroxyl group in an L- or D-β-arabinofuranosyl configuration.

The term “carbocyclic nucleoside” as used herein refers to a nucleoside or nucleoside analog containing a sugar-like moiety with 4′-oxo displaced by methylene or substituted methylene or methine such as cyclopentanes, cyclobutanes and the like, which are not N-glycosides and include phosphorylation site(s) or phosphate, or phosphonate, or their precursors.

The term “acyclic nucleoside” as used herein refers to a nucleoside or nucleoside analog containing an open partial sugar moiety, which includes phosphorylation site(s) or phosphate, or phosphonate, or their precursors.

The term “nucleophile” as used herein refers to an electron-rich reagent that is an electron pair donor (contains an unshared pair of electrons) and forms a new bond to a carbon atom. Nucleophiles can be anions or neutrally charged. Examples include, but are not limited to, carbanions, oxygen anions, halide anions, sulfur anions, nitrogen anions, nitrogen bases, imidazoles, pyridines, alcohols, ammonia, water, and thiols.

The term “leaving group” as used herein refers to a weakly basic chemical entity that is readily released from carbon, and takes the pair of bonding electrons binding it with said carbon atom. Leaving groups are chemical functional groups that can be displaced from carbon atoms by nucleophilic substitution. Examples include, but are not limited to heteroarenes including, but not limited to 1-N-alkylimidazole and pyridine, pentafluorophenol, halides including fluoride, chloride, bromide and iodide, alkylsulfonates, substituted alkylsulfonates, arylsulfonates, substituted arylsulfonates, heterocyclicsulfonates and trichloroacetimidate groups. Preferred leaving groups include, but are not limited to, chloride, bromide, iodide, p-nitrobenzenesulfonate (nosylate), p-(2,4-dinitroanilino)benzenesulfonate, benzenesulfonate, methylsulfonate (mesylate), p-methylbenzenesulfonate (tosylate), p-bromobenzenesulfonate (brosylate), trifluoromethylsulfonate (triflate), 2,2,2-trifluoroethanesulfonate, imidazolesulfonate, trichloroacetimidate, trifluoroacetate and other acylates, pentafluorophenoxide, and 2,4,6-trichlorophenoxide.

The terms “alkylating” and “alkylation” as used herein refer to formation of carbon-nitrogen bond by attack of N9 of purine to an electrophilic carbon center with a leaving group that is readily released from carbon, and takes the pair of bonding electrons binding it with said carbon atom. Examples of leaving groups include, but are not limited to, halides, sulfonates, oxophosphine, and trifluoroacetate and other acylates.

The term “glycosylating” and “glycosylation” as used herein refer to formation of carbon-nitrogen by attack of N9 of purine to an electrophilic carbon center with a leaving group that is readily released from carbon, and takes the pair of bonding electrons binding it with said carbon atom. The said carbon is directly bonded to another heteroatom such as O, S, Se, and NHR within a five- or six-membered ring.

The synonymous terms “hydroxyl protecting group” and “alcohol-protecting group” as used herein refer to substituents attached to the oxygen of an alcohol group commonly employed to block or protect the alcohol functionality while reacting other functional groups on the compound. Examples of such alcohol-protecting groups include but are not limited to the 2-tetrahydropyranyl group, 2-(bisacetoxyethoxy)methyl group, trityl group, trichloroacetyl group, carbonate-type blocking groups such as benzyloxycarbonyl, trialkylsilyl groups, examples of such being trimethylsilyl, tert-butyldimethylsilyl, tert-butyldiphenylsilyl, phenyldimethylsilyl, triiospropylsilyl and thexyldimethylsilyl, ester groups, examples of such being formyl, (C₁-C₁₀) alkanoyl optionally mono-, di- or tri-substituted with (C₁-C₆) alkyl, (C₁-C₆) alkoxy, halo, aryl, aryloxy or haloaryloxy, the aroyl group including optionally mono-, di- or tri-substituted on the ring carbons with halo, (C₁-C₆) alkyl, (C₁-C₆) alkoxy wherein aryl is phenyl, 2-furyl, carbonates, sulfonates, and ethers such as benzyl, p-methoxybenzyl, methoxymethyl, 2-ethoxyethyl group, etc. The choice of alcohol-protecting group employed is not critical so long as the derivatized alcohol group is stable to the conditions of subsequent reaction(s) on other positions of the compound of the formula and can be removed at the desired point without disrupting the remainder of the molecule. Further examples of groups referred to by the above terms are described by J. W. Barton, “Protective Groups In Organic Chemistry”, J. G. W. McOmie, Ed., Plenum Press, New York, N. Y., 1973, and G. M. Wuts, T. W. Greene, “Protective Groups in Organic Synthesis”, John Wiley & Sons Inc., Hoboken, N. J., 2007, which are hereby incorporated by reference. The related terms “protected hydroxyl” or “protected alcohol” define a hydroxyl group substituted with a hydroxyl protecting group as discussed above.

The term “nitrogen protecting group,” as used herein, refers to groups known in the art that are readily introduced on to and removed from a nitrogen atom. Examples of nitrogen protecting groups include but are not limited to acetyl (Ac), trifluoroacetyl, Boc, Cbz, benzoyl (Bz), N,N-dimethylformamidine (DMF), trityl, and benzyl (Bn). See also G. M. Wuts, T. W. Greene, “Protective Groups in Organic Synthesis”, John Wiley & Sons Inc., Hoboken, N.J. 2007, and related publications.

The term “acyl group” as used herein refers to a chemical entity comprising the general formula R—C(═O)— where R represents any aliphatic, alicyclic, or aromatic group and C(═O) represents a carbonyl group.

The term “acylation” as used herein refers to any process whereby an acid or an acid derivative such as an acid halide or an acid anhydride is used to convert a hydroxyl group into an ester, or an amine into an amide.

The terms “halogen” or “halo” as used herein refer to fluorine, chlorine, bromine and iodine; and the term “halide” refers to fluoride, chloride, bromide and iodide.

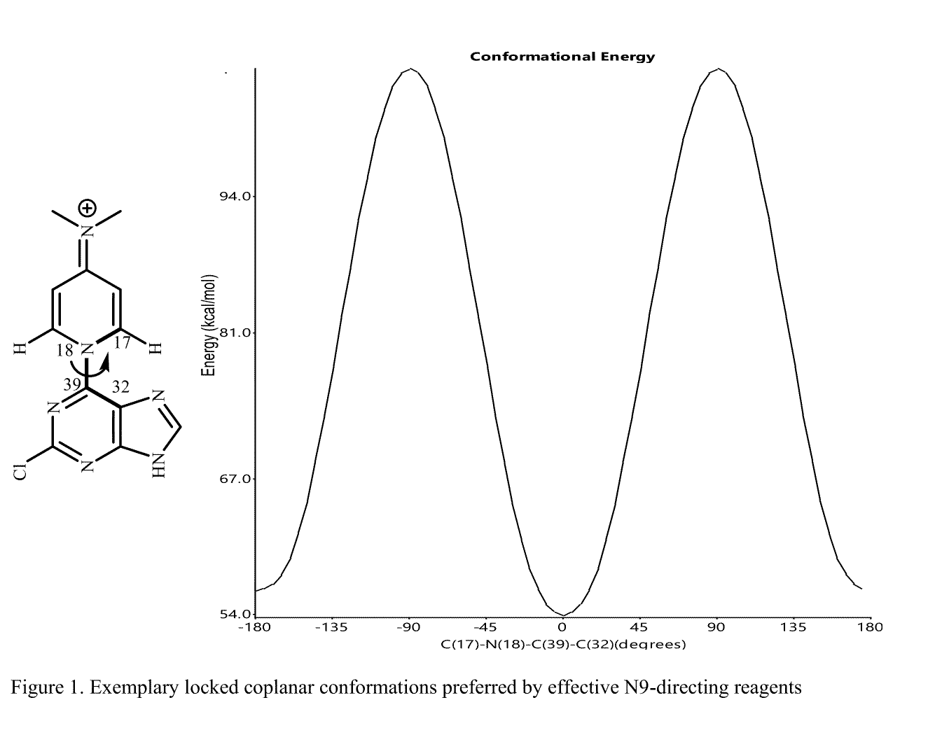

The term “locked coplanar conformation” as used herein refers to an energetically locked conformation that positions the 6-heteroaryl ring and purine ring in the same plane due to conjugation effects as showed in FIG. 1.

The terms “pyridyl”, “pyridinium”, “pyridin-1-yl”, and “4-aminopyridin-1-ium-1-yl” as used herein refer to nitrogenous aromatic compounds with 1) a “pyrrole-type” trivalent nitrogen atom in its 4-iminopyridine form or 4-oxopyridine form, 2) a “pyridine-type” aromatic trivalent nitrogen in its 4-aminopyridine form or 4-hydroxypyridine form, 3) a six-membered ring, and 4) aromaticity, including substituted and unsubstituted pyridine. 6-(4-Oxopyridinyl)purine (i.e. deprotonated 6-(4-hydroxylpyridinium-1-yl)purine), 6-pyridinium purine, and their substituted analogues (by 2-amino, 2-chloro, and etc.) are capable of excluding N7-alkylation and N7-glycosylation as presented in this invention.

The terms “imidazole”, “imidazolium”, “3-N-alkylimidazol-1-ium-1-yl”, “imidazolyl”, “1-N-alkylimidazol-3-yl”, and “3-N-alkylimidazol-1-yl” as used herein refer to nitrogenous aromatic compounds with 1) a “pyrrole-type” trivalent nitrogen atom which is alkylated or arylated, 2) a “pyridine-type” aromatic trivalent nitrogen with lone electron pair, which can be alkylated as an imidazolium salt, 3) a five-membered ring, and 4) aromaticity, including substituted and unsubstituted imidazole. 6-Imidazolyl purine and its substituted analogues (by 2-amino, 2-chloro, and etc.) were reported to exclude N7-alkylation and N7-glycosylation, and alkylation and glycoslylation of 6-imidazlium purines also give exclusively N-9 products as presented in this invention.

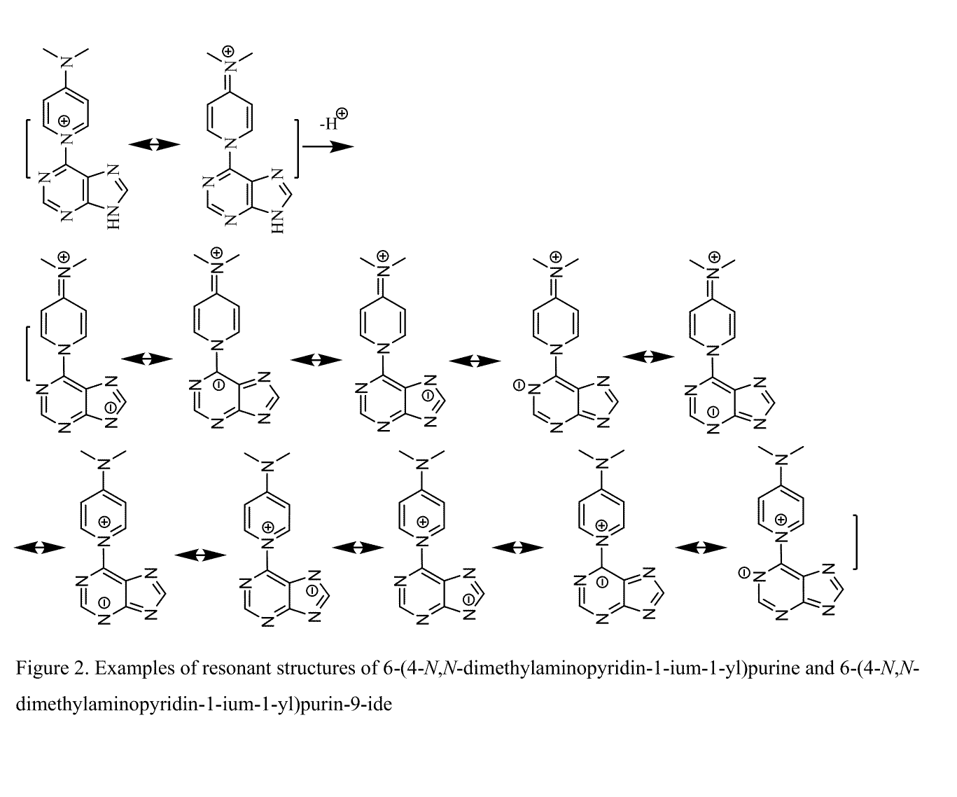

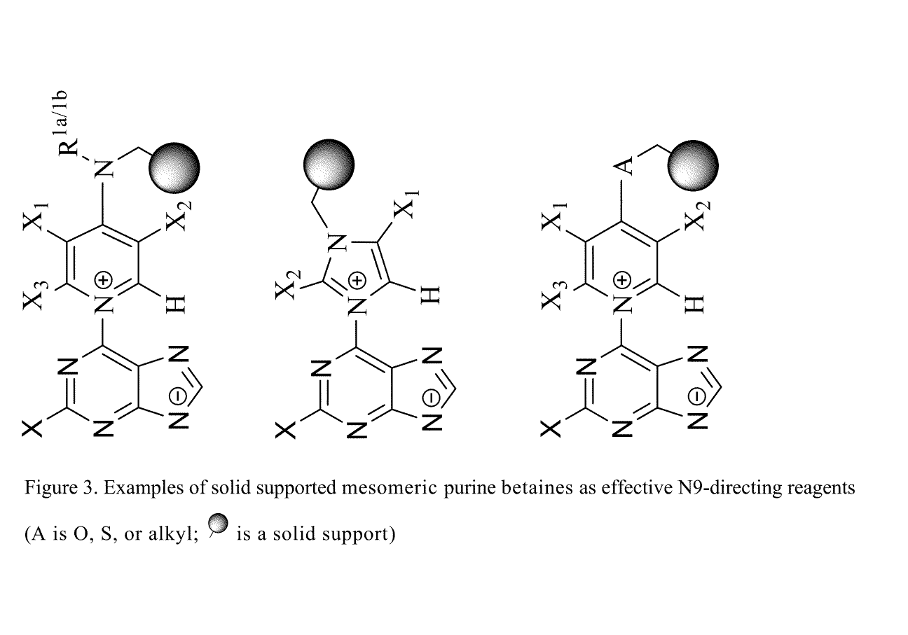

The terms “6-pyridylpurine”, “6-(pyridin-1-ium-1-yl)purine”, “6-(4-N,N-dialkylaminopyridin-1-ylpurine”, “6-(4-alkoxypyridin-1-ylpurine”, “6-(4-alkylthiopyridin-1-ylpurine”, “4-(dimethylamino)-1-(9H-purin-6-yl)pyridin-1-ium”, “6-(4-(dialkyliminio)pyridin-1(4H)-yl)purin-9-ide”, “6-(4-(dialkylamino)pyridin-1-ium-1-yl)purin-9-ide”, “3-N-alkyl-1-(purin-6-yl)imidazol-1-ium”, “6-(3-N-alkylimidazol-1-ium-1-yl)purin-9-ide”, and etc. as used herein refer to a resonant structure of a cationic or a neutral mesomeric 6-heteroarylpurine betaine, as showed by non-limiting examples of formulas for 6-pyridylpurines (FIG. 2). Examples of solid supported mesomeric purine betaines are given in FIG. 3.

The compounds of this invention and used or made in the methods of this invention can contain one or more asymmetric carbon atoms (chiral centers), so that the compounds can exist in different stereoisomeric forms. These compounds can be, for example, racemic mixtures, optically active non-racemic mixtures or diastereomers. In these situations, the single enantiomers, i.e., optically pure forms, can be obtained by asymmetric synthesis or by resolution of racemic mixtures. Resolution of racemic mixtures can be accomplished, for example, by conventional methods such as crystallization in the presence of a resolving agent; chromatography, using, for example a chiral HPLC column; or derivatizing the racemic mixture with a resolving reagent to generate diastereomers, separating the diastereomers via chromatography, and removing the resolving agent to generate the original compound in enantiomerically enriched form. Any of the above procedures can be repeated to increase the enantiomeric purity of a compound.

In one aspect of the present invention, a novel method for preparing N-9 purine nucleosides is provided. In one embodiment, the invention provides a method for preparing an N-9 purine nucleoside, comprising the steps of:
(a) glycosylating or alkylating a 6-(pyridine-1-ium-1-yl)purine or a 6-(3-N-alkylimidazol-1-ium-1-yl)purine at the N-9 position; and,
(b) displacing the pyridine or imidazole from the glycosylated or alkylated purine in step (a) with a nucleophile to yield an N-9 purine nucleoside.

In some embodiments, the method results in regiospecific glycosylation and alkylation.

In some embodiments, the method results in a substantially pure regioisomer. In some embodiments, the method results in a substantially pure regio- and stereoisomer.

In some embodiments, the nucleophile in step (b) is ammonia.

In other embodiments, the nucleophile in step (b) is a nitrogen-containing nucleophile that is converted into an amino substituent by a subsequent transformation (e.g., azide followed by reduction, benzylamine followed by hydrogenolysis, etc.), or forms a prodrug.

In other embodiments, the nucleophile in step (b) is an oxygen- or sulfur-nucleophile.

Another embodiment provides a method for preparing an N-9 purine nucleoside, comprising the steps of:
(a) introducing a pyridin-1-ium-1-yl group or an imidazol-1-ium-1-yl at the 6 position of a purine;
(b) glycosylating or alkylating the 6-(pyridin-1-ium-1-yl)purine product or the 6-(3-N-alkylimidazol-1-ium-1-yl)purine product from step (a) at the N-9 position; and,
(c) displacing the 6-(pyridin-1-ium-1-yl) group or 6-(imidazol-1-ium-1-yl) from step (a) with a nucleophile to yield an N-9 purine nucleoside.

In some embodiments, the method results in regiospecific glycosylation and alkylation.

In some embodiments, the method results in a substantially pure regioisomer. In some embodiments, the method results in a substantially pure regio- and stereoisomer.

In some embodiments, the nucleophile in step (c) is ammonia.

In other embodiments, the nucleophile in step (c) is a nitrogen-containing nucleophile that is converted into an amino substituent by a subsequent transformation (e.g., azide followed by reduction, benzylamine followed by hydrogenolysis, etc.) or forms a prodrug.

In other embodiments, the nucleophile in step (c) is an oxygen- or sulfur-nucleophile.

In some embodiments a pyridin-1-ium-1-yl substituent or a 3-N-alkylimidazol-1-ium-1-yl is introduced at the 6 position of the purine by contacting the purine with a pyridine or a 1-N-alkylimidazole under nucleophilic displacement conditions.

Suitable agents for introducing such a group on to a 6-substituted purine with a leaving group at the 6 position include substituted pyridines, 4-aminopyridines, 4-alkoxypyridines, 4-alkylthiopyridines, and 1-N-alkylimidazoles. Nucleophilic displacement reactions preferably transpire in polar unreactive solvents such as dimethylformamide or acetonitrile at 15 to 135° C.

In some embodiments, the glycosylation step is performed by contacting a glycosylating agent in an unreactive solvent with a zwitterionic 6-(pyridin-1-ium-1-yl)purin-9-ide or 6-(3-N-alkylimidazol-1-ium-1-yl)purin-9-ide salt.

In some embodiments, the glycosylation step is performed by contacting a glycosylating agent in an unreactive solvent with a 6-(pyridin-1-ium-1-yl)purine or a 6-(3-N-alkylimidazol-1-ium-1-yl)purine at the presence of a Lewis acid such as SnCl₄ or SbCl₄.

In some embodiments, the glycosylation step is performed by contacting a glycosylating agent in an unreactive solvent with a persilylated 6-(pyridin-1-ium-1-yl)purin-9-ide or a persilylated 6-(3-N-alkylimidazol-1-ium-1-yl)purin-9-ide at the presence of a Lewis acid such as TMSOTf.

Suitable glycosylation agents for glycosylating a 6-(pyridin-1-ium-1-yl)purine and a 6-(3-N-alkylimidazol-1-ium-1-yl)purine include, but are not limited to, pentofuranoses, 2-deoxypentofuranoses, 3-deoxypentofuranoses, 2,3-dideoxypentofuranoses, substituted pentofuranoses, substituted 2-deoxypentofuranoses, substituted 3-deoxypentofuranoses and substituted 2,3-dideoxypentofuranoses, and analogs of the above classes of carbohydrate derivatives with a sulfur or selenium atom in place of the furanosyl ring oxygen atom, all with protected alcohol (OH) groups. Preferably, the activated sugar is selected from a group consisting of activated and O-protected sugars including, but not limited to, 2,3,5-tri-O-acetyl-β-D- or or L-ribofuranosyl chloride, 2,3,5-tri-O-benzoyl-β-D- or L-ribofuranosyl bromide, 2-deoxy-3,5-di-O-p-toluoyl-α-D- or L-erythro-pentofuranosyl chloride, 3-deoxy-2,5-di-O-benzoyl-β-D- or L-erythro-pentofuranosyl chloride, 2-deoxy-2-fluoro-3,5-di-O-benzoyl-α-D- or L-arabinofuranosyl bromide, 2,3-dideoxy-2-fluoro-5-O-p-toluoyl-α-D- or L-glycero-pentofuranosyl chloride (also called 2,3-dideoxy-2-fluoro-5-O-p-toluoyl-α-D- or L-arabinofuranosyl chloride), 2-deoxy-2,2-difluoro-3,5-di-O-benzoyl-β-D- or L-erythro-pentofuranosyl triflate and 2,3,5-tri-O-benzyl-α-D- or L-arabinofuranosyl bromide, and their analogs with a sulfur or selenium atom in place of the furanosyl ring oxygen atom. In these embodiments, chloride, bromide, and triflate are leaving groups. Other leaving groups may be substituted for the chloride or bromide leavings groups including, but not limited to, fluoride, iodide, mesylate, tosylate, trichloroacetimidate, acetate, benzoate and other acylates, etc. Other hydroxyl protecting groups, which are familiar to anyone skilled in the art, may be substituted for the indicated acetyl, benzoyl, p-toluoyl, etc. groups.

Suitable glycosylating or alkylating agents may also be represented by Formula VIII

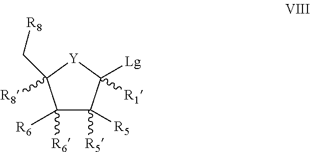

in which Lg is a leaving group or a group in-situ convertible to a leaving group; R₅, R₆, R₈, R_{1′}, R_{5′}, R_{6′}, and R_{8′} are each independently selected from hydrogen, protected hydroxyl, halogen including fluoro, chloro, bromo and iodo, cyno, alkynyl, alkyl (C₁-C₆), substituted alkyl, alkoxyl (C₁-C₆), protected nitrogen; R_{5′}, R_{6′}, and R_{8′} can be linked in such a way to form a bicyclic sugar analogue; and Y is a CR₂, oxygen, sulfur (or oxosulfur with Lg selected as hydrogen), selenium (or oxoselenium with Lg selected as hydrogen), or a nitrogen atom with a bonded hydrogen atom, an alkyl (C₁-C₆) or an acyl group.

Glycosylations preferably are carried out using glycosylating agents with transiently protected hydroxyl groups.

Glycosylations without neighboring group participation preferably are performed in the presence of a base in solutions of mixed solvents with a minimum amount of a more polar (higher dielectric constant) unreactive solvent such as acetonitrile or dimethylformamide to increase the solubility of the zwitterionic 6-(pyridin-1-ium-1-yl)purin-9-ide salt or 6-(3-N-alkylimidazol-1-ium-1-yl)purin-9-ide salt, and a less polar (lower dielectric constant) unreactive solvent such as chloroform, dichloromethane, tetrahydrofuran or toluene. The protected and activated sugar derivative can be soluble in the less polar solvent and the low polarity (lower dielectric constant) of that solvent strongly retards ionization of the glycosyl-leaving group bond thus minimizing anomerization of the activated sugar derivative and maximizing formation of the desired nucleoside diastereoisomer.

Alternatively, these glycosylation reactions may be performed in a single solvent. Glycosylations may also be performed in three or more solvents to fine-tune the polarity and preferential solvation characteristics of the combination. Preferably, the solvents of single and multiple solvent combinations are anhydrous.

These glycosylations preferably transpire with an inner salt of a 6-(pyridin-1-ium-1-yl)purin-9-ide or of a 6-(3-N-alkylimidazol-1-ium-1-yl)purin-9-ide, initially formed in situ by treatment of the 6-(pyridin-1-ium-1-yl)purine or 6-(3-N-alkylimidazol-1-ium-1-yl)purine salt, with an appropriate counter ion such as halides and sulfates, with a hydride base such as sodium hydride or potassium hydride, a strong base such as sodium hexamethyldisilazide or potassium hexamethyldisilazide, or alkaline metal carbonates such as sodium carbonate, potassium carbonate, and cesium carbonate. Glycosylations carried out in polar solvent systems can solubilize partially or fully the resultant inner salt of a 6-(pyridin-1-ium-1-yl)purin-9-ide or a 6-(3-N-alkylimidazol-1-ium-1-yl)purin-9-ide.

Optionally, strong bases with both organic cation and anion components may be used to enhance the solubility of the resulting purine salt in non-polar solvents. When strong bases with organic cation and anion components are used, glycosylations with a zwitterionic 6-(pyridin-1-ium-1-yl)purin-9-ide salt or 6-(3-N-alkylimidazol-1-ium-1-yl)purin-9-ide salt may be carried out in a single solvent of low polarity.

Optionally, catalysts such as sodium iodide can be included. The glycosylations can be conducted at temperatures from about 0° C. to about 50° C.

Glycosylations with neighboring group participation preferably are performed in the presence of a Lewis acid with 6-(pyridin-1-ium-1-yl)purines, or 6-(3-N-alkylimidazol-1-ium-1-yl)purines, or their persilylated derivatives.

Alkylations preferably are performed in the presence of a base or bases, a catalyst such as Pd(PPh₃)₄, or under Mitsunobu reaction condition.

Optionally, alkylations or glycosylations may be performed on solid supports for library and combinatorial synthesis of nucleosides based on 6-(pyridin-1-ium-1-yl)purines or 6-(3-N-alkylimidazol-1-ium-1-yl)purines bound to solid supports via a covalent linker at 6-heteroarylium.

In some embodiments following glycosylation or alkylation, concomitant displacement of the 6-(pyridin-1-ium-1-yl) group or a 6-(3-N-alkylimidazol-1-ium-1-yl) group and any O-protection groups occurs by direct ammonolysis at the 6-position.

In some embodiments following glycosylation or alkylation, displacement of the appended 6-(pyridin-1-ium-1-yl) ring or 6-(3-N-alkylimidazol-1-ium-1-yl) ring by a hydroxide nucleophile gives the corresponding 6-oxopurine compound.

In some embodiments, concomitant displacement of the 6-(pyridin-1-ium-1-yl) group or a 6-(3-N-alkylimidazol-1-ium-1-yl) group and any O-protection groups occurs by base-promoted hydrolysis.

In some embodiments following glycosylation, displacement of the appended 6-(pyridin-1-ium-1-yl) ring or 6-(3-N-alkylimidazol-1-ium-1-yl) ring by a nitrogen-, oxygen- or sulfur-based nucleophile gives the corresponding 6-(substituted-amino)-, 6-(disubstituted-amino)-, 6-(substituted-oxy)- or 6-(substituted-sulfanyl)purine compound in which the substituents on nitrogen, oxygen, or sulfur are chosen from groups including, but not limited to, hydrogen, alkyl (C₁-C₆), aryl, heteroaryl, and arylalkyl. In some embodiments, concomitant displacement of the 6-heteroaryl group and any O-protection groups occurs.

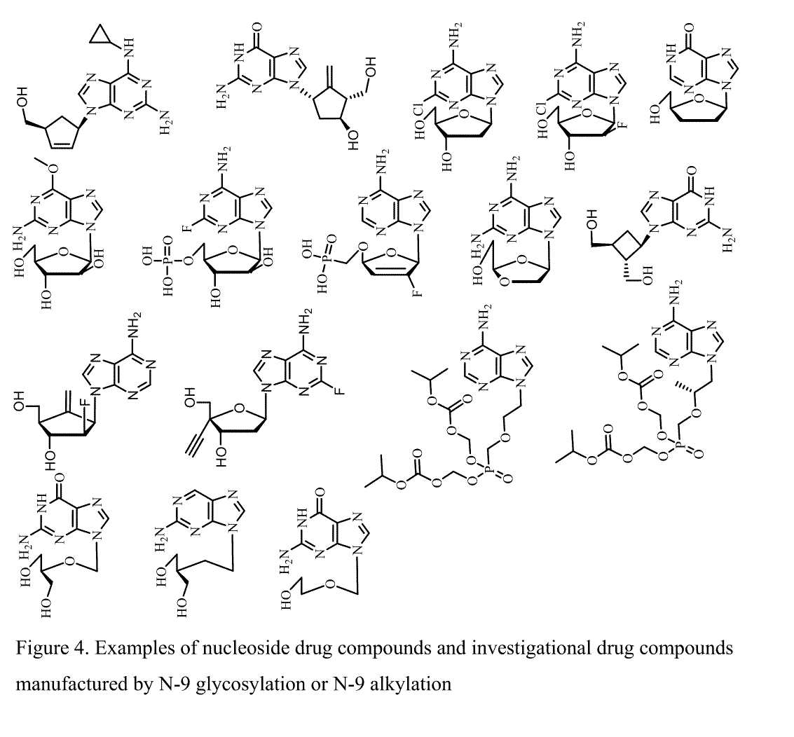

The N-9 regiospecific glycosylation and alkylation methods provide efficient access to 9-β-D- or L-purine nucleosides, including the adenosines, guanosines, inosines and substituted derivatives thereof, deoxynucleosides including the deoxyadenosines, deoxyguanosines, deoxyinosines and substituted derivatives thereof, acyclic nucleosides, and carbocyclic nucleosides. Specific nucleosides and deoxynucleosides include, but are not limited to, the 2′-deoxyadenosines, 2′-deoxy-2′-α- or β-halogenated-deoxyadenosines, 3′-deoxyadenosines, 2′,3′-dideoxyadenosines, 2′-deoxy-2′-β-F-adenosines (such as 2-chloro-2′-deoxy-2′-F-araA, Clofarabine), Abacavir, Entecavir, Nelarabine, 2′,3′-dideoxy-2′-β-F-adenosines, adenine arabinosides such as adenine arabinoside (araA) and 2-F-araA (Fludarabine) and the like (FIG. 4).

In one embodiment, a method of regiospecific N-9 glycosylation of purines comprises contacting a substituted purine of Formula I

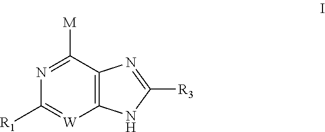

with a base in a more polar solvent, and treating the resulting inner salt with a glycosylating or an alkylating agent of the formula R₆-Lg wherein W is selected from —N—, —CH— and CR₂, and M is a substituted pyridinium or a substituted imidazolium

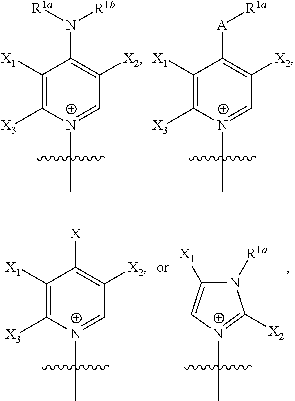

, where R₁, R₂, R₃, R^{1a}, R^{1b}, A, X, X₁, X₂, and X₃ are as defined above, preferably with X₃ selected as hydrogen, and where R₆ is a glycosyl or an alkyl group, and Lg is a leaving group. The resulted protected nucleoside may be subjected to ammonolysis to obtain nucleosides of Formula IX.

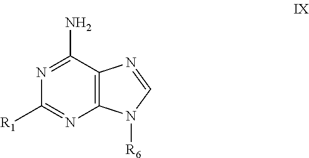

In one such process, 9-β-D- or L-purine 2′-deoxynucleosides, including the deoxyadenosines, are prepared by glycosylating a mesomeric purine betaine derived from a purine having the Formula X

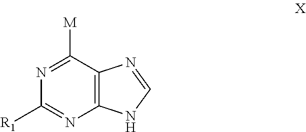

with 2-deoxy-3,5-di-O-p-toluoyl-α-D- or L-erythro-pentofuranosyl chloride as the glycosylating agent. The resulting compound of Formula XI

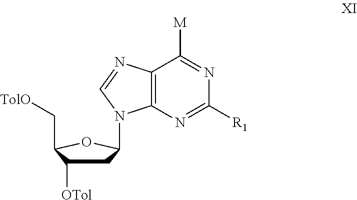

can be deprotected by ammonolysis at C-6 and of the alcohol protecting groups resulting in formation of the 2′-deoxynucleosides of Formula XII.

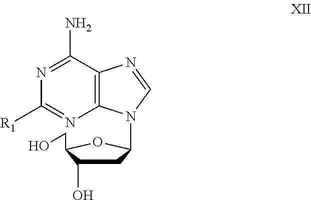

In one such process, the glycosylating agent is 3-deoxy-2,5-di-O-benzoyl-α-D- or L-erythro-pentofuranosyl chloride, glycosylation results in the compound with Formula XIII.

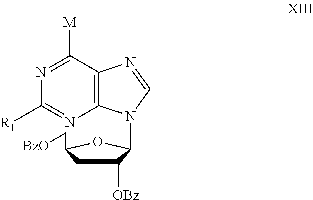

The compound of Formula XIII can be deprotected by ammonolysis at C-6 and of the alcohol protecting groups resulting in formation of the 3′-deoxynucleosides of Formula XIV.

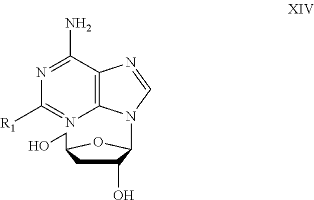

In one such process, the glycosylating agent is 3,5-di-O-benzoyl-2-deoxy-2-fluoro-α-D- or L-arabinofuranosyl bromide, glycosylation results in formation of the compound with Formula XV.

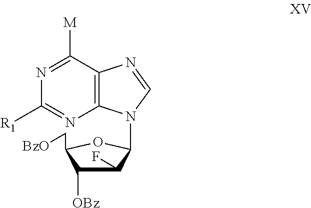

The compound of Formula XV can be deprotected by ammonolysis at C-6 and of the alcohol protecting groups resulting in formation of the 2′-deoxy-2′-fluoro arabino nucleosides of Formula XVI.

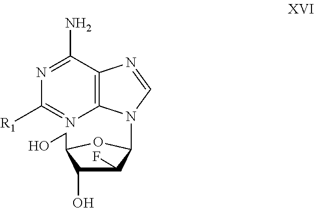

In one such process, the glycosylating agent is 2,3-dideoxy-2-fluoro-5-O-p-toluoyl-α-D- or L-threo-pentofuranosyl chloride, glycosylation results in formation of the compound with Formula XVII.

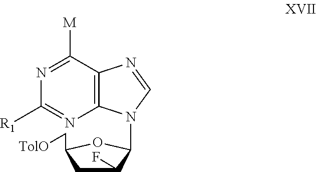

The compound of Formula XVII can be deprotected by ammonolysis at C-6 and of the alcohol protecting groups resulting in formation of the 2′,3′-dideoxy-2′-fluoro threo nucleosides of Formula XVIII.

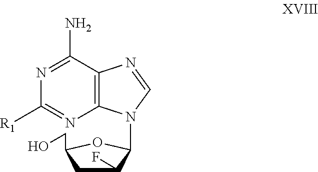

In one such process, the glycosylating agent is 2,3,5-tri-O-benzyl-α-D- or L-arabinofuranosyl bromide, glycosylation results in formation of the compound with Formula XIX.

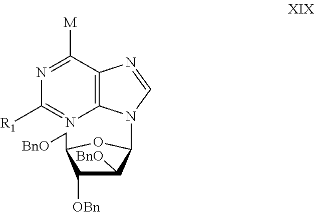

The compound of Formula XIX can be deprotected by ammonolysis at C-6 and removal of the alcohol protecting groups resulting in formation of the nucleoside of Formula XX,

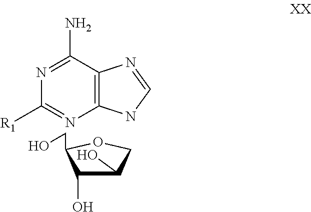

or by methanolysis at C-6 and removal of the alcohol protecting groups resulting in formation of the nucleoside of Formula XXI.

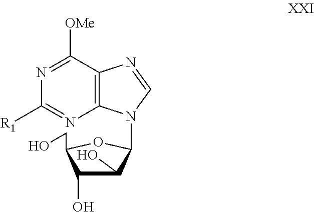

In one such process, the glycosylating agent is 3,5-di-O-benzoyl-2-deoxy-2-fluoro-2-methyl-α-D- or L-ribofuranosyl bromide, glycosylation results in formation of the compound with Formula XXII.

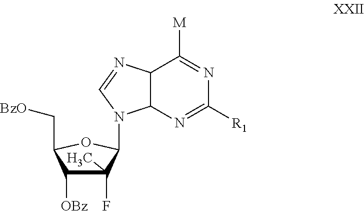

The compound of Formula XXII can be transformed resulting in formation of the nucleoside of Formula XXIII.

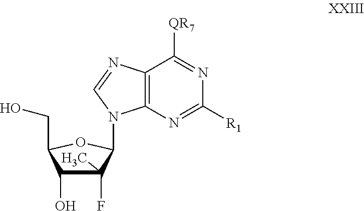

In one such process, the glycosylating agent is 2,3,5-tri-O-benzoyl-2-methyl-α-D- or L-ribofuranosyl bromide, glycosylation results in formation of the compound with Formula XXIV.

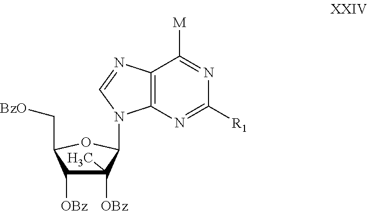

The compound of Formula XXIV can be transformed resulting in formation of the nucleoside of Formula XXV.

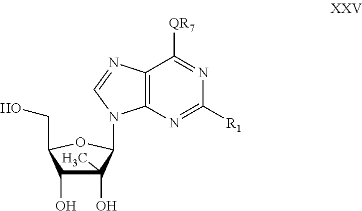

In one such process, the alkylating agent is a substituted cyclopentane, alkylation results in formation of the compounds with Formula including, but not limited to, XXVI-XXX.

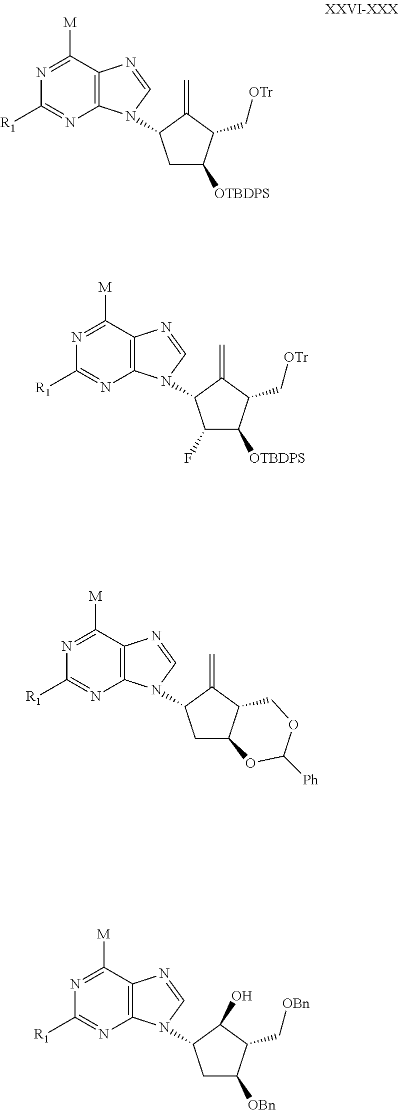

The compounds of Formula XXVI-XXX can be transformed resulting in formation of the nucleoside of Formula XXXI

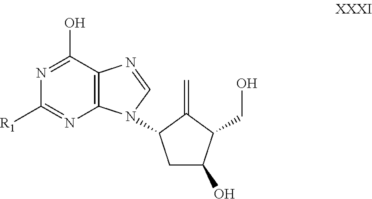

or the nucleoside of Formula XXXII.

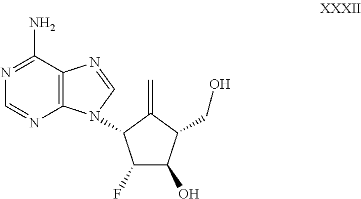 [SMILES: C=C1[C@@H](n2cnc3c(N)ncnc32)[C@@H](F)[C@H](O)[C@H]1CO]

In one such process, the alkylating agent is a substituted cyclopentene, alkylation results in formation of the compound with Formula XXXIII.

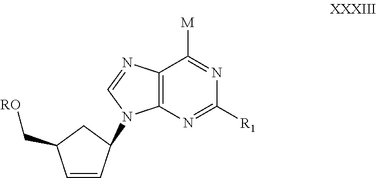

The compound of Formula XXXIII can be transformed resulting in formation of the nucleoside of Formula XXXIV.

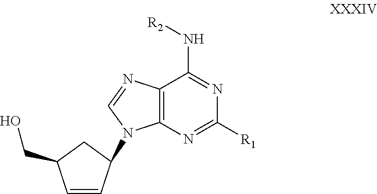

Yet in another such process, the alkylating agent is a substituted cyclobutane, alkylation results in formation of the compound with Formula XXXV.

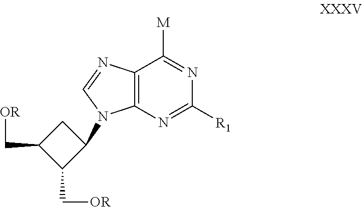

The compound of Formula XXXV can be transformed resulting in formation of the nucleoside of Formula XXXVI.

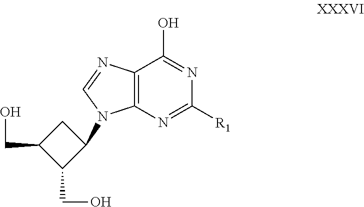

Some embodiments of N-9 alkylation are given in Scheme 1.

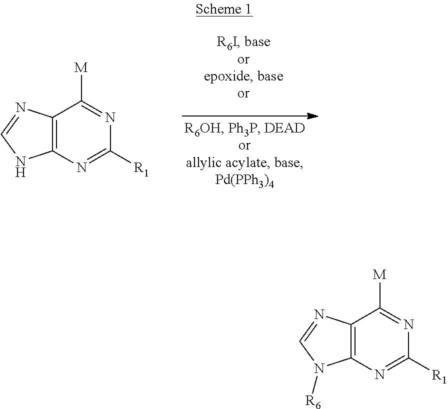

In other embodiments, the inner salts of the 2-chloro-6-(pyridin-1-ium-1-yl)purin-9-ides or the 2-chloro-6-(3-N-alkylimidazol-1-ium-1-yl)purin-9-ides can be coupled with 2-deoxy-3,5-di-O-(p-toluoyl)-α-D-erythro-pentofuranosyl chloride according to Scheme 2. In preferred embodiments, the glycosylation is carried out in binary solvent mixtures with the more polar (higher dielectric constant) solvent used to solubilize the purine salt and the non-polar solvent (low dielectric constant) used to dissolve the sugar derivative and minimize anomerization of the glycosyl halide.

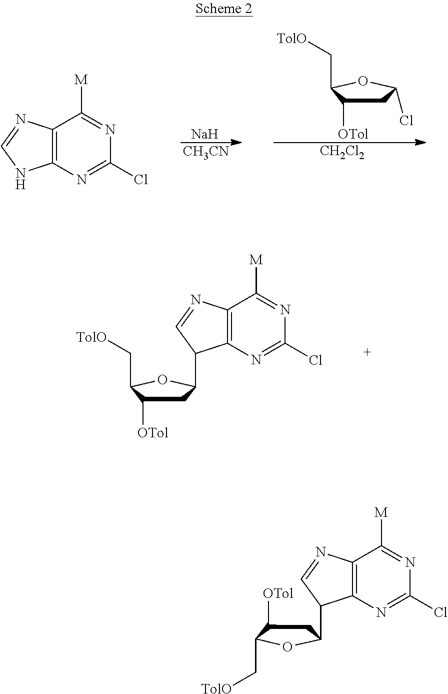

The β anomers from Scheme 2 can form Cladribine product by ammonolysis at C-6 and of the alcohol protecting groups according to Scheme 3.

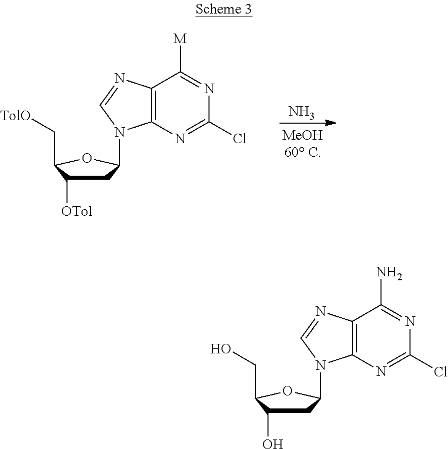

In one example, a method for the preparation of 2-CdA (cladribine) comprises:
(a) contacting a 6-substituted purine compound having Formula III:

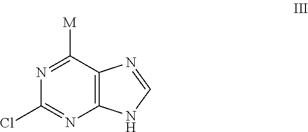

with a base in a first polar solvent followed by contacting an activated and hydroxyl-protected 2-deoxy-α-D-erythro-pentofuranosyl compound in a second less polar solvent to form a nucleoside product,
(b) contacting the protected nucleoside from step (a) with ammonia in a third solvent to obtain 2-CdA.

In some of the embodiments, the first polar solvent is a single solvent or a binary solvent mixture with an average dielectric constant of between about 5 and about 40. In other embodiments, the first polar solvent has an average dielectric constant of about 20. In some embodiments, the first polar solvent is acetonitrile. In other embodiments, the first polar solvent is a mixture of acetonitrile and dichloromethane. In other embodiments, the first polar solvent is a mixture of three solvents such as acetonitrile, acetone, tetrahydrofuran, dioxane, and the like.

In some embodiments with a 6-(pyridine-1-ium-1-yl) substituent or a 6-(3-N-alkylimidazol-1-ium-1-yl) substituent, the corresponding heteroarylium is introduced at C-6 of the purine by contacting a purine derivative with a pyridine or a 1-N-alkylimidazole under nucleophilic displacement conditions. The leaving group can already be in place at C-6 or can be generated in situ in the reaction medium.

In some embodiments with a 6-pyridinium substituent or a 6-imidazolium substituent on solid supports, the corresponding heteroarylium is introduced at C-6 of the purine by contacting a purine derivative with a pyridine or an imidazole on solid supports under nucleophilic displacement conditions.

Suitable agents for introduction of a pyridine at C-6 of a purine with a leaving group already at the 6 position include, but are not restricted to, substituted and unsubstituted pyridines, 4-(N,N-dialkylamino)pyridines, 1-N-alkylimidazoles, 4-alkoxylpyridines, and 4-alkylthiopyridines. Nucleophilic displacement reactions preferably transpire in polar unreactive solvents such as dimethylformamide, acetonitrile, or in neat corresponding heteroarenes at about 15° C. to about 135° C.

The cationic 6-heteroarylpurines can be prepared from 6-chloropurines according to procedures shown in Scheme 4.

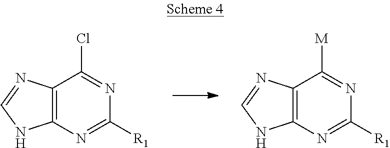

In yet another aspect of the invention, novel cationic 6-heteroarylpurine compounds and corresponding neutral mesomeric 6-heteroarylpurine betaines after deprotonation of purine are provided. The 6-heteroaryl groups are useful for directing regiospecific N-9 alkylation and glycosylation reactions. The formed cationic nucleoside intermediates include 6-heteroaryl as good leaving groups which are activated by the N-9 alkylation or N-9 glycosylation, and are easily displaced with various nucleophiles to provide therapeutic agents.

In one example, a compound of Formula I is provided

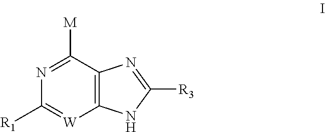

where W is selected from —N—, —CH— and CR₂, and M is a substituted pyridine or a substituted imidazole

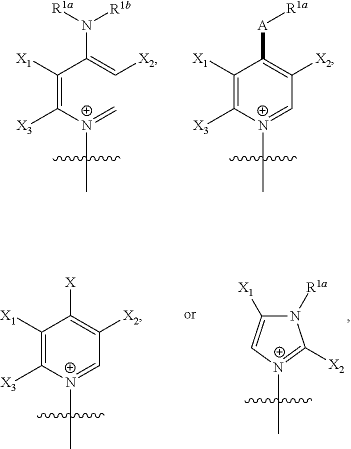

and where R₁, R₂, R₃, X, X₁, X₂, R^{1a} and R^{1b}, and X₃ are selected as follows: (i) each R₁, R₂, R₃, X, X₁, X₂, and X₃ is independently selected from hydrogen, C₁₋₁₀ alkyl, C₁₋₁₀ alkoxy, C₁₋₁₀ alkylthio, halogen, amino, hydroxyl, C₁₋₁₀ alkylamino, di-C₁₋₁₀ alkylamino, C₁₋₁₀ acylamino, trialkylsilyl, aryl, and heteroaryl, preferably with X₃ selected as hydrogen; (ii) R^{1a} and R^{1b} is independently selected from C₁₋₁₀ alkyl, aryl, and heteroaryl; (iii) R^{1a}R^{1b}, X₁, X₂ together form cyclic rings, preferably with X₃ selected as hydrogen; (iv) R^{1a}, R^{1b}, X, X₁, X₂ or X₃ is bound to solid supporting polymers; and where A is O, S, or alkyl; and pharmaceutically acceptable salts of these compounds.

### Examples

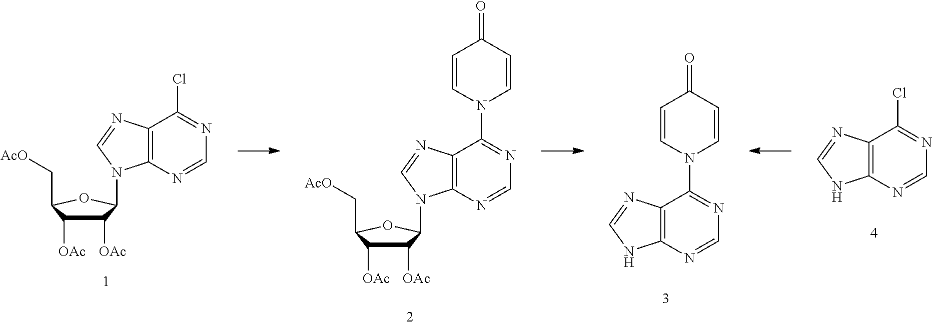

### Preparation of 6-(4-oxopyridin-1-yl)purine

### Method 1. Synthesis of 6-Pyridylpurine by Deglycosylation of Nucleosides

Step a (Method 1): A mixture of 9-(2,3,5-tri-O-acetyl-β-D-ribofuranosyl)-6-chloropurine (7.8 g, 19.0 mmol), 4-hydroxypyridine (9.0 g, 95.0 mmol), and DIPEA (4.93 g, 6.6 mL, 38 mmol) was dissolved in CH₃CN (100 mL) and stirred at reflux (about 110° C.) under N2 for 16 h (reaction was complete, TLC). After removal of volatiles, the residue was chromatographed (CH₂Cl₂ then MeOH/CH₂Cl₂, 1:19) to give 9-(2,3,5-tri-O-acetyl-β-D-ribofuranosyl)-6-(4-oxopyridin-1-yl)purine (10.45 g, 117%) including DIPEA salt: ¹H NMR (500 MHz, CDCl₃) δ 9.37 (d, J=2.2 Hz, 2H), 8.82 (s, 1H), 8.31 (s, 1H), 6.57 (d, J=2.2 Hz, 2H), 6.28 (d, J=5.1 Hz, 1H), 5.96 (t, J=5.3 Hz, 1H), 5.66 (t, J=5.2 Hz, 1H), 4.52-4.49 (m, 1H), 3.70-3.64 (m, 1H), 3.13-3.08 (m, 1H), 2.18 (s, 3H), 2.15 (s, 3H), 2.10 (s, 3H).

Step a (Method 2): A mixture of 4-hydroxypyridine (0.21 g, 2.2 mmol) and sodium hydride (60% dispersion in mineral oils, 107 mg, 2.66 mmol) in DMF (5 mL) was stirred at rt under nitrogen for 17 min. The resulting mixture was added to cold 9-(2,3,5-tri-O-acetyl-β-D-ribofuranosyl)-6-chloropurine (0.82 g, 2.0 mmol) in DMF (5 mL) stirred at 0° C. under nitrogen. The reaction was stirred at 0° C. for 2 h, TLC analysis showed complete reaction). After removal of volatiles, the residue was chromatographed (CH₂Cl₂ then MeOH/CH₂Cl₂, 1:13) to give 9-(2,3,5-tri-O-acetyl-β-D-ribofuranosyl)-6-(4-pyridin-1-yl)purine (0.766 g, 82%).

Step b: 9-(2,3,5-Tri-O-acetyl-β-D-ribofuranosyl)-6-(4-pyridin-1-yl)purine (2.7 g, 5.7 mmol, contaminated with DIPEA salt) was dissolved in IPA (190 mL). To the solution was added AcCl (1.9 mL, 2.10 g, 26.7 mmol), and the mixture was stirred at 65° C. for 23 h in a sealed steel vessel. The reaction was cooled to rt, and solid precipitation was observed. Volatiles were evaporated in vacuo, and the residue was washed with DCM (50 mL) by stirring for 10 min. The solid was then collected by filtration, washed with DCM twice, and dried under vacuum to give the title compound (1.02 g, 102%, crude as a salt): ¹H NMR (500 MHz, DMSO-d₆) δ 9.45 (d, J=6.8 Hz, 2H), 8.89 (s, 1H), 8.77 (s, 1H), 6.71 (d, J=6.0 Hz, 2H).

Method 2. To a mixture of hydroxypyridine (2.03 g, 21.4 mmol) and sodium hydride (60% dispersion in mineral oils, 0.94 g, 23.5 mmol) was carefully added DMF (30 mL), and the resulting slight suspension was stirred at rt under nitrogen for 30 min. To this reaction mixture was then added 6-chloropurine (2.98 g, 19.4 mmol), and the reaction was stirred at rt under nitrogen for 18 h. No significant reaction was detected by TLC analysis. The reaction temperature was then elevated to 135° C., and heavy precipitation was observed in 2.5 h. The reaction was stirred at that temperature for another 5.5 h, TLC analysis showed complete reaction). The reaction mixture was poured into water (100 mL), and the solid was collected by filtration. This solid was further washed with water (30 mL) and methanol (30 mL×2) and dried under vacuum to give 6-(4-oxopyridin-1-yl)purine (2.26 g, 53%): ¹H NMR (500 MHz, DMSO-d₆) δ 9.31 (d, J=8.1 Hz, 2H), 8.81 (s, 1H), 8.69 (s, 1H), 6.41 (d, J=8.1 Hz, 2H).

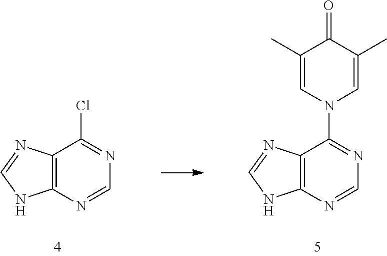 [SMILES: Clc1ncnc2[nH]cnc12 ; Cc1cn(-c2ncnc3[nH]cnc23)cc(C)c1=O]

### Preparation of 6-(3,5-dimethyl-4-oxopyridin-1-yl)purine

A mixture of 6-chloropurine (0.46 g, 3.0 mmol), 4-hydroxy-3,5-dimethylpyridine (0.55 g, 4.5 mmol), and DIPEA (1.16 g, 1.6 mL, 9.0 mmol) was dissolved in CH₃CN (20 mL) and stirred at reflux (about 110° C.) under N₂ for 16 h. After removal of volatiles, the residue was precipitated in water (20 mL), and the solid was collected by filtration, and washed thoroughly with acetone to provide 5 as a solid (92 mg, 13%): ¹H NMR (500 MHz, DMSO-d₆) δ 9.28 (2H), 8.80 (s, 1H), 8.70 (s, 1H), 2.00 (s, 6H).

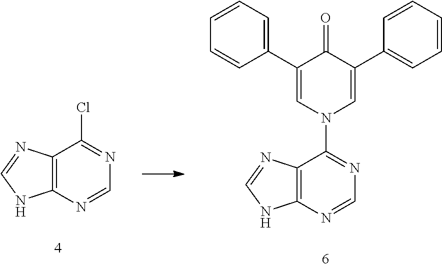 [SMILES: Clc1ncnc2[nH]cnc12 ; O=c1c(-c2ccccc2)cn(-c2ncnc3[nH]cnc23)cc1-c1ccccc1]

### Preparation of 6-(4-oxo-3 5-diphenylpyridin-1-yl)purine

To a mixture of 4-hydroxy-3,5-diphenylpyridine (1.09 g, 4.4 mmol) and sodium hydride (60% dispersion in mineral oils, 0.19 g, 4.8 mmol) was added DMF (20 mL), and the resulting slight suspension was stirred at rt under nitrogen for 1 h. The reaction temperature was then elevated to 135° C., and 6-chloropurine (0.62 g, 4.0 mmol) was added, and the reaction was stirred at 135° C. under nitrogen for 4 h (TLC analysis showed nearly complete reaction). The reaction mixture was cooled room temperature, concentrated till dryness, and MeOH-DCM (1:2, 30 mL) was added. The resulting solution was passed through a short silica gel plug, and the plug column was rinsed with MeOH-DCM (1:2 to 1:1) till very slight UV spot. All fractions were then combined, and concentrated till heavy yellow precipitation. The solid was collected by filtration, washed with methanol (10 mL×2), and dried under vacuum to give 6-(4-oxo-3,5-diphenylpyridin-1-yl)purine (0.64 g, 40%): ¹H NMR (500 MHz, DMSO-d₆) δ 9.53 (2H), 8.88 (s, 1H), 8.74 (s, 1H), 7.71-7.38 (m, 10H).

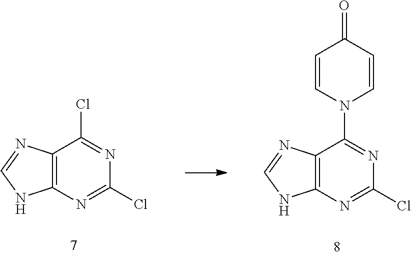 [SMILES: Clc1nc(Cl)c2nc[nH]c2n1 ; O=c1ccn(-c2nc(Cl)nc3[nH]cnc23)cc1]

### Preparation of 2-chloro-6-(4-oxopyridin-1-yl)purine

Method 1. A mixture of 2,6-dichloropurine (2.0 g, 10.6 mmol) and 4-hydroxypyridine (6.0 g, 63.5 mmol) in DMF (36 mL) was stirred under nitrogen at 65° C. for 23 h. Pink heavy precipitation was observed, and TLC analysis showed nearly complete reaction. The reaction was cooled to rt, and poured into water (50 mL/g), and the precipitated solid was collected by filtration, washed with water and methanol, and dried under vacuum to give the title compound (2.38 g, 76%): ¹H NMR (500 MHz, DMSO-d₆) δ 9.17 (d, J=8.0 Hz, 2H), 8.73 (s, 1H), 6.42 (d, J=8.2 Hz, 2H).

Method 2. To a mixture of hydroxypyridine (1.11 g, 11.6 mmol) and sodium hydride (60% dispersion in mineral oils, 0.51 g, 12.8 mmol) was carefully added DMF (10 mL), and the resulting slight suspension was stirred at rt under nitrogen for 30 min. To this reaction mixture was then added 2,6-dichloropurine (2.0 g, 10.6 mmol), and the reaction was stirred at rt under nitrogen for 18 h. No significant reaction was detected by TLC analysis. The reaction temperature was then elevated to 135° C., and heavy precipitation was observed in 2.5 h. The reaction was stirred at that temperature for another 5.5 h, TLC analysis showed complete reaction). The reaction mixture was cooled to rt, poured into water (50 mL), and the solid was collected by filtration. This solid was further washed with water (15 mL) and methanol (15 mL x 2) and dried under vacuum to give 2-chloro-6-(4-oxopyridin-1-yl)purine (2.27 g, 75%).

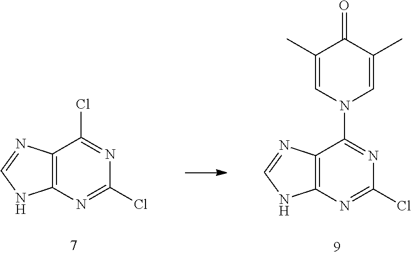 [SMILES: Clc1nc(Cl)c2nc[nH]c2n1 ; Cc1cn(-c2nc(Cl)nc3[nH]cnc23)cc(C)c1=O]

### Preparation of 2-chloro-6-(3,5-dimethyl-4-oxopyridin-1-yl)purine

A mixture of 2,6-dichloropurine (1.02 g, 5.5 mmol), 4-hydroxy-3,5-dimethylpyridine (1.11 g, 8.3 mmol), and DIPEA (2.13 g, 2.9 mL, 16.5 mmol) was dissolved in CH₃CN (30 mL) and stirred at reflux (about 110° C.) under N₂ for 21 h. After removal of volatiles, the residue was precipitated in water (50 mL), and the solid was collected by filtration, and washed thoroughly with acetone to provide 9 as a solid (1.03 g, 68%): ¹H NMR (500 MHz, DMSO-d₆) δ 9.09 (2H), 8.66 (s, 1H), 1.96 (s, 6H).

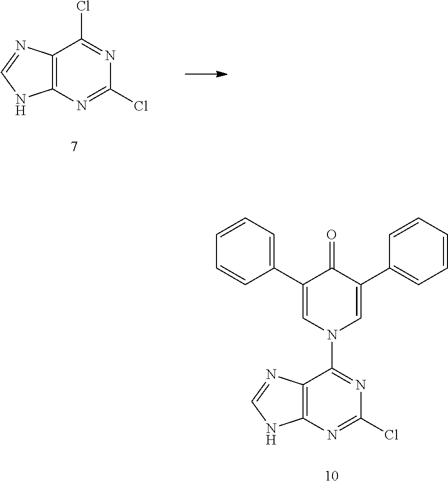 [SMILES: Clc1nc(Cl)c2nc[nH]c2n1 ; O=c1c(-c2ccccc2)cn(-c2nc(Cl)nc3[nH]cnc23)cc1-c1ccccc1]

### Preparation of 2-chloro-6-(4-oxo-3,5-diphenylpyridin-1-yl)purine

To a mixture of 4-hydroxy-3,5-diphenylpyridine (1.09 g, 4.4 mmol) and sodium hydride (60% dispersion in mineral oils, 0.19 g, 4.8 mmol) was carefully added DMF (20 mL), and the resulting slight suspension was stirred at rt under nitrogen for 5.5 h. The reaction was then cooled to 0° C., and 2,6-dichloropurine (0.76 g, 4.0 mmol) was added, and the reaction was stirred at rt under nitrogen for 2 h. The reaction temperature was then elevated, and the reaction was stirred at 135° C. under nitrogen for 17 h (TLC analysis showed nearly complete reaction). The reaction mixture was concentrated till dryness, and MeOH-DCM (1:2, 30 mL) was added. The resulting solution was passed through a short silica gel plug, and the plug column was rinsed with MeOH-DCM (1:2 to 1:1) till very slight UV spot. All fractions were then combined, and concentrated till heavy yellow precipitation. The solid was collected by filtration, washed with methanol (10 mL×2), and dried under vacuum to give 2-chloro-6-(4-oxo-3,5-diphenylpyridin-1-yl)purine (1.03 g, 64%): ¹H NMR (500 MHz, DMSO-d₆) δ 9.47 (2H), 8.51 (s, 1H), 7.69-7.38 (m, 10H).

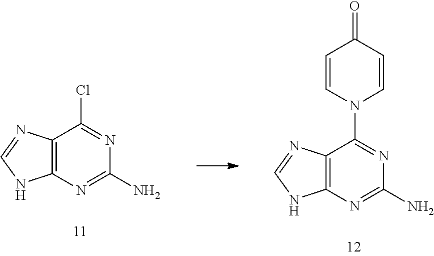 [SMILES: Nc1nc(Cl)c2nc[nH]c2n1 ; Nc1nc(-n2ccc(=O)cc2)c2nc[nH]c2n1]

### Preparation of 2-amino-6-(4-oxopyridin-1-yl)purine

A mixture of 2-amino-6-chloropurine (2.0 g, 11.8 mmol) and 4-hydroxypyridine (6.73 g, 70.8 mmol) in DMF (36 mL) was stirred under nitrogen at 65° C. for 20 h. Pink heavy precipitation was observed. The reaction was cooled to rt, and poured in to cold water (400 mL). The solid was collected by filtration, washed in water (50 mL×2) and ethyl ether (30 mL), and dried under vacuum. TLC analysis showed a slight bright smeared spot at the bottom. This solid was then transferred to a 50 mL RBF. 4-Hydroxypyrine (3.4 g, 35.8 mmol) was added followed by DMF (30 mL). This gave an initially clean solution, followed by precipitation. The mixture was stirred at 130° C. for 3 days, then cooled to rt, and poured in to water (100 mL). The solid was collected by filtration, washed with water and methanol, and dried under vacuum to give a solid (2.1 g). TLC analysis showed it still included some pyridine, which was removed by washing with methanol to give the title compound (2.06 g, 77%): ¹H NMR (500 MHz, DMSO-d₆) δ 9.19 (d, J=8.1 Hz, 2H), 8.18 (s, 1H), 6.73 (s, 2H), 6.36 (d, J=8.1 Hz, 2H).

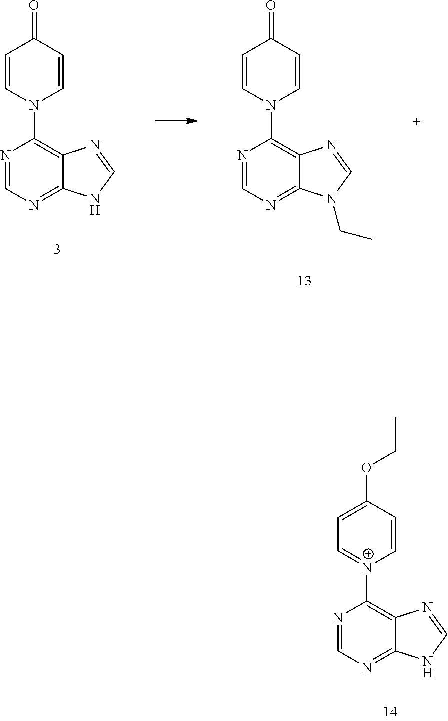 [SMILES: O=c1ccn(-c2ncnc3[nH]cnc23)cc1 ; CCn1cnc2c(-n3ccc(=O)cc3)ncnc21 ; CCOc1cc[n+](-c2ncnc3[nH]cnc23)cc1]

### Preparation of 9-ethyl-6-(4-oxopyridin-1-yl)purine

A mixture of 6-(4-oxopyridin-1-yl)purine (0.5 g, 2 mmol) and sodium hydride (60% w/w suspension, 0.18 g, 4.4 mmol) in anhydrous DMF (10 mL) was stirred at ambient temperature under N₂ for 40 min. Ethyl iodide (0.94 g, 0.48 mL, 6 mmol) was added dropwise with a syringe. The reaction mixture was then stirred for 18 h. TLC analysis showed a very minor spot for 6-EtO by-product, and a low spot for an O-alkylated byproduct, but no N7 isomer was formed. The reaction mixture was analyzed by ¹H NMR to show both N9- and O-alkylation (7:1). Volatiles were evaporated in vacuo, and the residue was coated onto silica gel and separated by column chromatograph (43 g silica gel, CH₂Cl₂ then MeOH/CH₂Cl₂, 1:11) to give the title compound [0.28 g, 58% (HCl form assumed for s.m.)]: ¹H NMR (500 MHz, DMSO-d₆) δ 9.41 (d, J=8.2 Hz, 2H), 8.82 (s, 1H), 6.56 (d, J=8.2 Hz, 2H), 4.42 (d, J=7.3 Hz, 2H), 1.63 (d, J=7.4 Hz, 3H).

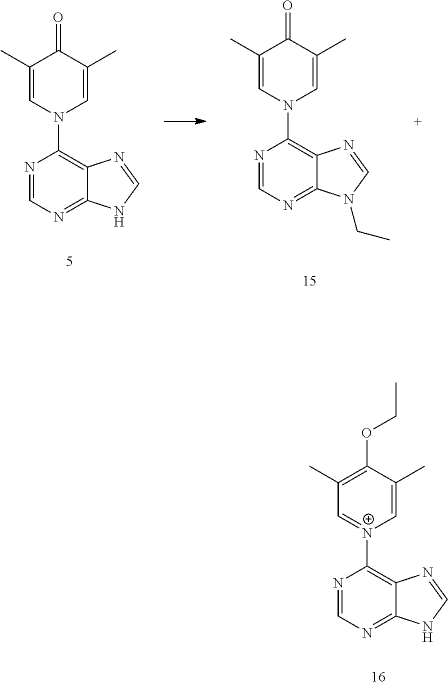 [SMILES: Cc1cn(-c2ncnc3[nH]cnc23)cc(C)c1=O ; CCn1cnc2c(-n3cc(C)c(=O)c(C)c3)ncnc21 ; CCOc1c(C)c[n+](-c2ncnc3[nH]cnc23)cc1C]

### Preparation of 9-ethyl-6-(3,5-dimethyl-4-oxopyridin-1-yl)purine

A mixture of 6-(3,5-dimethyl-4-oxopyridin-1-yl)purine (72 mg, 0.3 mmol) and K₂CO₃ (62 mg, 0.45 mmol) in anhydrous DMF (5 mL) was stirred at ambient temperature under N₂. Ethyl iodide (70 mg, 0.036 mL, 0.45 mmol, diluted in DMF) was added dropwise with a syringe. The reaction mixture was stirred for 18 h. Volatiles were evaporated in vacuo, and the residue was analyzed by ¹H NMR to show both N9- and O-alkylation (5.4:1).

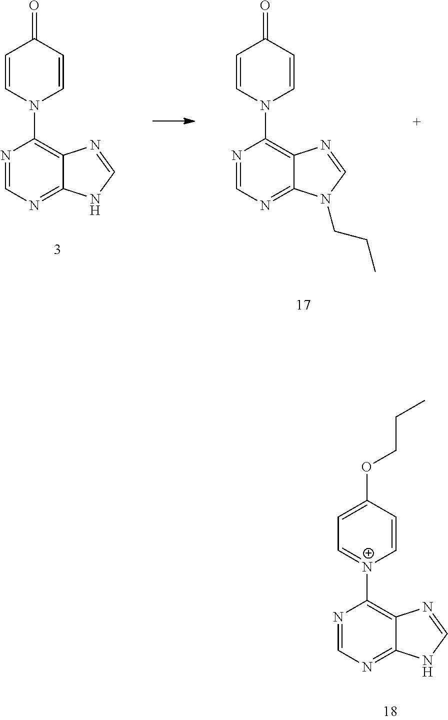 [SMILES: O=c1ccn(-c2ncnc3[nH]cnc23)cc1 ; CCCn1cnc2c(-n3ccc(=O)cc3)ncnc21 ; CCCOc1cc[n+](-c2ncnc3[nH]cnc23)cc1]

### Preparation of 9-propyl-6-(4-oxopyridin-1-yl)purine

A mixture of 6-(4-oxopyridin-1-yl)purine (106 mg, 0.5 mmol) and K₂CO₃ (62 mg, 0.45 mmol) in anhydrous DMF (5 mL) was stirred at ambient temperature under N2. Propyl iodide (125 mg, 0.072 mL, 0.75 mmol, diluted in DMF) was added dropwise with a syringe. The reaction mixture was then stirred for 18 h at rt, and then at 50° C. for 30 h. Volatiles were evaporated in vacuo, and the residue was coated onto silica gel and separated by column chromatograph (43 g silica gel, CH₂Cl₂ then MeOH/CH₂Cl₂, 1:30 to 1:5) to give 17 (128 mg): ¹H NMR (500 MHz, DMSO-d₆) δ 9.45 (d, J=8.5 Hz, 2H), 9.10 (s, 1H), 8.40 (s, 1H), 6.43 (d, J=8.5 Hz, 2H), 4.30 (t, J=7.3 Hz, 2H), 1.89 (sext, J=7.3 Hz, 2H), 0.87 (t, J=7.4 Hz, 3H). Also isolated a byproduct formed by alkylation at exocyclic oxygen of pyridine ring, 18 (25 mg): ¹H NMR (500 MHz, DMSO-d₆) δ 9.28 (d, J=8.5 Hz, 2H), 8.86 (s, 1H), 8.79 (s, 1H), 6.41 (d, J=8.5 Hz, 2H), 4.59 (t, J=7.3 Hz, 2H), 1.96 (sext, J=7.3 Hz, 2H), 0.91 (t, J=7.4 Hz, 3H).

The byproduct was treated with 0.2 M NaOMe-MeOH (5.0 eq.) to provide a mesomeric betaine.

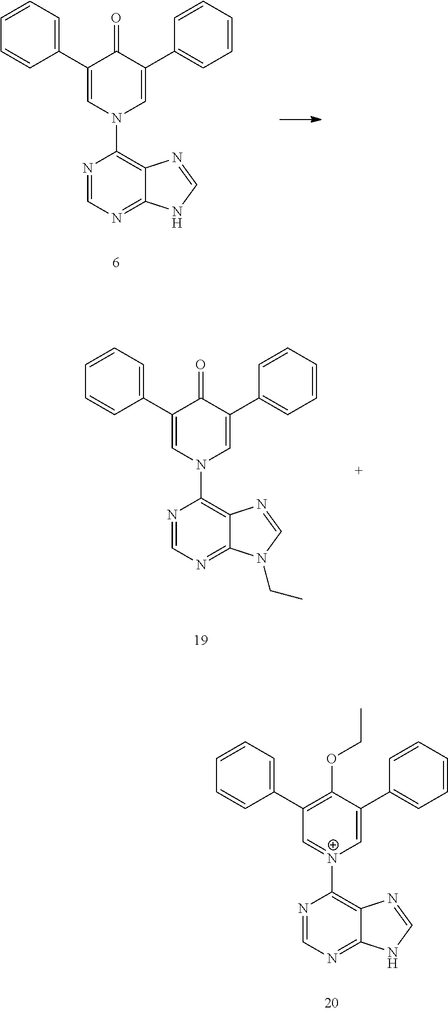 [SMILES: O=c1c(-c2ccccc2)cn(-c2ncnc3[nH]cnc23)cc1-c1ccccc1 ; CCn1cnc2c(-n3cc(-c4ccccc4)c(=O)c(-c4ccccc4)c3)ncnc21 ; CCOc1c(-c2ccccc2)c[n+](-c2ncnc3[nH]cnc23)cc1-c1ccccc1]

### Preparation of 9-ethyl-6-(3,5-diphenyl-4-oxopyridin-1-yl)purine

A mixture of 6-(3,5-diphenyl-4-oxopyridin-1-yl)purine (91 mg, 0.25 mmol) and K₂CO₃ (52 mg, 0.38 mmol) in anhydrous DMF (5 mL) was stirred at ambient temperature under N₂. Ethyl iodide (58 mg, 0.03 mL, 0.38 mmol, diluted in DMF) was added dropwise with a syringe. The reaction mixture was stirred at rt for 18 h. Volatiles were evaporated in vacuo, and the resulting residue was dissolved in DCM (30 mL), washed with saturated NaHCO₃ aqueous solution (20 mL), and dried form anhydrous MgSO₄. Volatiles were evaporated, and the residue was analyzed by ¹H NMR to show both N9- and O-alkylation (7:1) with N9-as the major product: ¹H NMR (500 MHz, DMSO-d₆) δ 9.49 (2H), 8.92 (s, 1H), 8.81 (s, 1H), 7.49-7.28 (m, 10H), 4.38 (q, J=7.3 Hz, 2H), 1.48 (t, J=7.4 Hz, 3H).

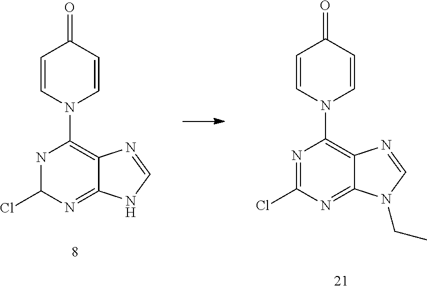 [SMILES: O=c1ccn(C2=c3nc[nH]c3=NC(Cl)N2)cc1 ; CCn1cnc2c(-n3ccc(=O)cc3)nc(Cl)nc21]

### Preparation of 2-chloro-9-ethyl-6-(4-oxopyridin-1-yl)purine

A mixture of 2-chloro-6-(4-oxopyridin-1-yl)purine (0.20 g, 0.81 mmol), ethyl iodide (0.38 g, 0.2 mL, 2.42 mmol) and K₂CO₃ (0.34 g, 2.42 mmol) in anhydrous DMF (10 mL) was stirred at ambient temperature under N₂ for 20 h. TLC analysis showed nearly complete reaction to form essentially N9-product with no N7 product detected. Solid was then removed by filtration. Volatiles of filtrate were evaporated in vacuo and the residue was chromatographed (23 g silica gel, CH₂Cl₂ then MeOH/CH₂Cl₂, 1:15) to give the title compound (0.216 g, 97%): ¹H NMR (500 MHz, CDCl₃) δ 9.35 (d, J=8.1 Hz, 2H), 8.13 (s, 1H), 6.55 (d, J=8.2 Hz, 2H), 4.38 (t, J=7.3 Hz, 2H), 1.61 (t, J=7.3 Hz, 3H).

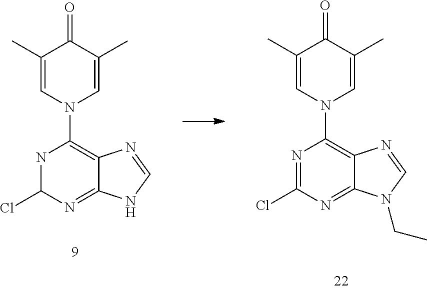 [SMILES: Cc1cn(C2=c3nc[nH]c3=NC(Cl)N2)cc(C)c1=O ; CCn1cnc2c(-n3cc(C)c(=O)c(C)c3)nc(Cl)nc21]

### Preparation of 2-chloro-9-ethyl-6-(3,5-dimethyl-4-oxopyridin-1-yl)purine

A mixture of 2-chloro-6-(3,5-dimethyl-4-oxopyridin-1-yl)purine (0.20 g, 0.81 mmol), ethyl iodide (0.38 g, 0.2 mL, 2.42 mmol) and K₂CO₃ (0.34 g, 2.42 mmol) in anhydrous DMF (10 mL) was stirred at ambient temperature under N2 for 20 h. TLC analysis showed nearly complete reaction. Solid was then removed by filtration. Volatiles were evaporated in vacuo, and the resulting residue was dissolved in DCM (30 mL), washed with saturated NaHCO₃ aqueous solution (20 mL), and dried form anhydrous MgSO₄. Volatiles were evaporated, and the residue was analyzed by ¹H NMR to show essentially N9-product: ¹H NMR (500 MHz, CDCl₃) δ 9.13 (s, 2H), 8.77 (s, 1H), 4.30 (d, J=8.2 Hz, 2H), 1.45 (t, J=7.3 Hz, 3H).

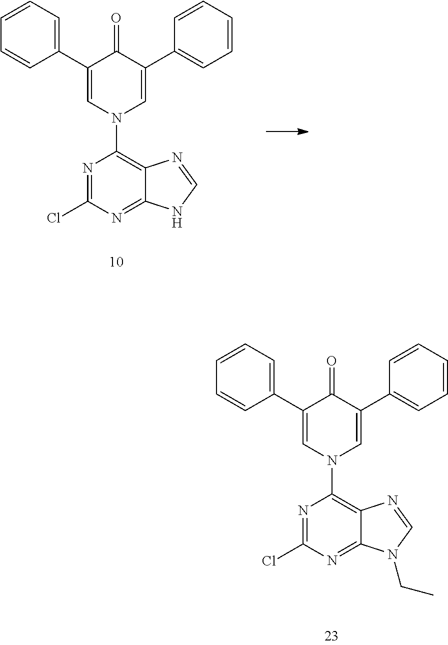 [SMILES: O=c1c(-c2ccccc2)cn(-c2nc(Cl)nc3[nH]cnc23)cc1-c1ccccc1 ; CCn1cnc2c(-n3cc(-c4ccccc4)c(=O)c(-c4ccccc4)c3)nc(Cl)nc21]

### Preparation of 2-chloro-9-ethyl-6-(4-oxo-3,5-diphenylpyridin-1-yl)purine

A mixture of 2-chloro-6-(4-oxo-3,5-diphenylpyridin-1-yl)purine (100 mg, 0.25 mmol), ethyl iodide (58 mg, 0.03 mL, 0.38 mmol) and K₂CO₃ (52 mg, 0.38 mmol) in anhydrous DMF (5 mL) was stirred at ambient temperature under N₂ for 20 h. TLC analysis showed nearly complete reaction. Volatiles were evaporated in vacuo, and the resulting residue was dissolved in DCM (30 mL), washed with saturated NaHCO₃ aqueous solution (20 mL), and dried form anhydrous MgSO₄. Volatiles were evaporated, and the residue was analyzed by ¹H NMR to show essentially N9-product (90 mg): ¹H NMR (500 MHz, CDCl₃) δ 9.34 (2H), 8.82 (s, 1H), 7.68-7.39 (m, 10H), 4.33 (q, J=7.3 Hz, 2H), 1.46 (t, J=7.4 Hz, 3H)

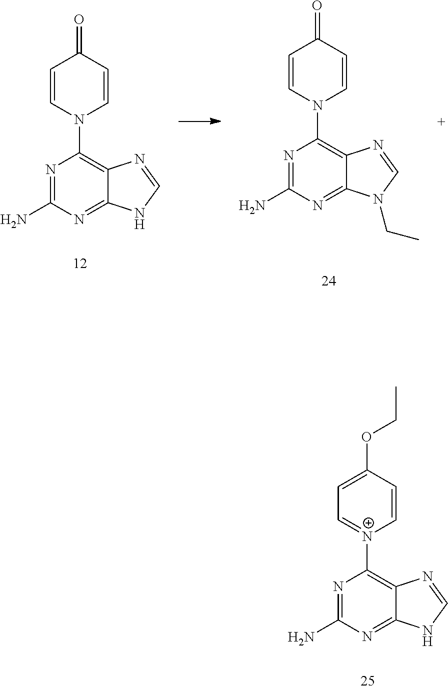 [SMILES: Nc1nc(-n2ccc(=O)cc2)c2nc[nH]c2n1 ; CCn1cnc2c(-n3ccc(=O)cc3)nc(N)nc21 ; CCOc1cc[n+](-c2nc(N)nc3[nH]cnc23)cc1]

### Preparation of 2-amino-9-ethyl-6-(4-oxopyridin-1-yl)purine

A mixture of 2-amino-6-(4-oxopyridin-1-yl)purine (0.23 g, 1.0 mmol), propyl iodide (0.3 mL, 3.0 mmol) and K₂CO₃ (0.414 g, 3.0 mmol) in anhydrous DMF (10 mL) was stirred at ambient temperature under N₂ for 16 h. The reaction temperature was elevated to 65° C., and stirred at 65° C. for 24 h. TLC analysis showed nearly complete reaction with extra spots above the product spot. Volatiles of filtrate were evaporated in vacuo and the residue was chromatographed (30 g silica gel, CH₂Cl₂ then MeOH/CH₂Cl₂, 1:10) to give the title compound (309 mg, including solvents (H₂O and MeOH) and some impurities, but no N7 regioisomer): ¹H NMR (500 MHz, CDCl₃) δ 9.29 (d, J=8.1 Hz, 2H), 7.79 (s, 1H), 6.53 (d, J=8.2 Hz, 2H), 5.04 (bs, 2H), 4.11 (t, J=7.2 Hz, 2H), 1.93 (sextet, J=7.3 Hz, 2H), 1.00 (t, J=7.4 Hz, 3H). Also isolated a byproduct formed by alkylation at exocyclic oxygen of pyridine ring.

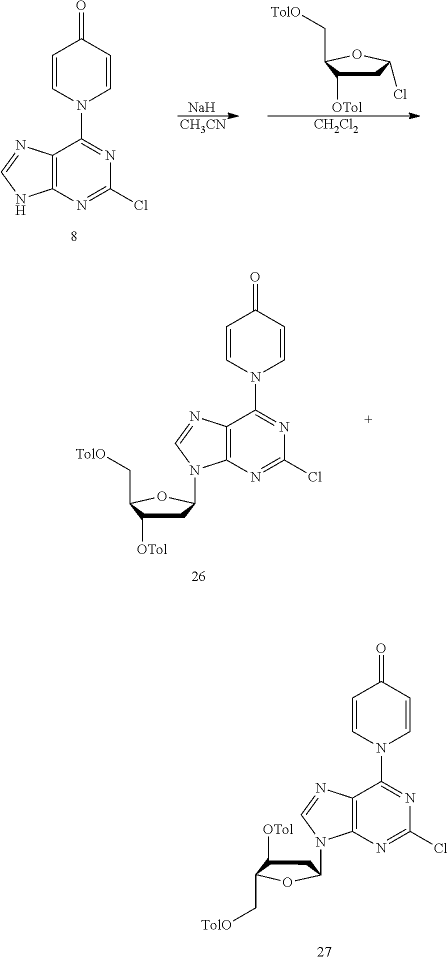

### Glycosylation of 2-chloro-6-(4-oxopyridin-1-yl)purine

A mixture of 2-chloro-6-(4-oxopyridin-1-yl)purine (0.50 g, 2.0 mmol) and sodium hydride (60% w/w suspension, 97 mg, 2.4 mmol) in dried CH₃CN (20 mL) was stirred at ambient temperature under N₂ for 15.5 h. The solution was chilled to 0° C., and a solution of 2-deoxy-3,5-di-O-(p-toluoyl)-α-D-erythro-pentofuranosyl chloride (0.9 g, 2.5 mmol) in cold, dried CH₂Cl₂ (20 mL, C) was added with a syringe. The reaction mixture was then stirred 0° C. for 2 h, and allowed to gradually warm to ambient temperature over 2 h. Then acetone (10 mL) was added, and the reaction was stirred for another hour, and TLC analysis showed minor unreacted purine. Suspension was removed by filtration, and filtrate was concentrated till dryness to give a yellow residue, which was dissolved in DCM (20 mL) and the insoluble solid of small amount was removed by filtration. Concentration of filtrate to give a yellow foam (1.33 g). This residue was chromatographed (44 g silica gel, MeOH/CH₂Cl₂, 1:90 to MeOH/CH₂Cl₂, 1:30) to give 2-chloro-6-(4-oxopyrindinyl)-2′-deoxy-3′,5′-di-O-(p-toluoyl)-α/β-D-erythro-pentofuranosylpurine as a mixture of two anomers (1.07 g, 88%, α/β1:1.7).

2-chloro-6-(4-oxopyridin-1-yl)-2′-de oxy-3′,5′-di-O-(p-toluoyl)-α-D-erythro-pentofuranosylpurine: ¹H NMR (500 MHz, CDCl₃) δ 9.31 (d, J=8.2 Hz, 2H), 8.44 (s, 1H), 7.97 (d, J=8.3 Hz, 2H), 7.57 (d, J=8.2 Hz, 2H), 7.30 (d, J=8.5 Hz, 2H), 7.16 (d, J=8.5 Hz, 2H), 6.66 (dd, J=5.6, 2.7 Hz, 1H), 6.56 (d, J=8.2 Hz, 2H), 5.71-5.73 (m, 1H), 4.97-4.99 (m, 1H), 4.61-4.68 (m, 2H), 3.10-3.08 (m, 2H), 2.45 (s, 3H), 2.37 (s, 3H).

2-chloro-6-(4-oxopyridin-1-yl)-2′-deoxy-3′,5′-di-O-(p-toluoyl)-β-D-erythro-pentofuranosylpurine: ¹H NMR (500 MHz, CDCl₃) δ 9.27 (d, J=8.2 Hz, 2H), 8.30 (s, 1H), 7.99 (d, J=8.2 Hz, 2H), 7.82 (d, J=8.2 Hz, 2H), 7.31 (d, J=8.5 Hz, 2H), 7.18 (d, J=8.5 Hz, 2H), 6.59 (“t”, J=6.8 Hz, 1H), 6.55 (d, J=8.2 Hz, 2H), 5.80-5.83 (m, 1H), 4.85 (dd, J=12.1, 3.5 Hz, 1H), 4.71-4.75 (m, 1H), 4.66 (dd, J=12.1, 4.0 Hz, 1H), 3.10-3.03 (m, 2H), 2.46 (s, 3H), 2.37 (s, 3H).

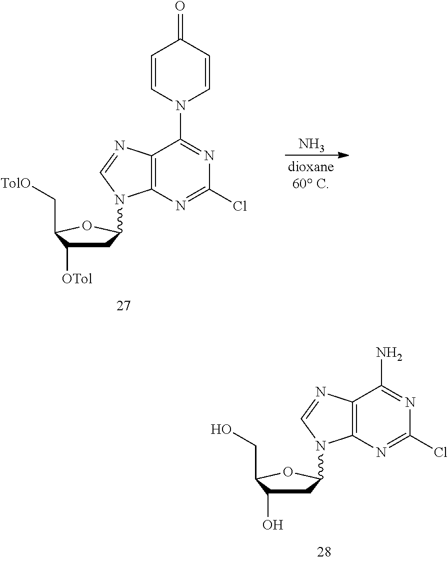

### Aminolysis of 2-chloro-6-(4-oxopyridin-1-yl)-2′-deoxy-3′ 5′-di-O-(p-toluoyl)-D-erythro-pentofuranosylpurines

2-Chloro-6-(4-oxopyridin-1-yl)-[2′-deoxy-3′,5′-di-O-(p-toluoyl)-α/β-D-erythro-pentofuranosyl]purine (1.0 g, crude) in NH₃/MeOH (7 M, 30 mL) in a sealed 100 mL pressure tube was stirred at 60° C. for 42 h, and TLC analysis showed nearly complete reaction. The reaction was stirred at 60° C. for additional 21 h, and TLC showed complete reaction. Volatiles were evaporated, and the residue was dissolved in 1:1 methanol/DCM, and coated onto limited amount of silica gel. Column chromatography (silica gel, 32 g, MeOH/DCM, 1:6) gave a mixture of two anomers (0.29 g, 61%): cladribine, ¹H NMR (500 MHz, DMSO-d₆) δ 8.35 (s, 1H), 7.81 (br, 2H), 6.26 (t, J=6.7 Hz, 1H), 5.30 (d, J=4.3 Hz, 1H), 4.97 (t, J=5.6 Hz, 1H), 4.38 (s, 1H), 3.85 (s, 1H), 3.58-3.60 (m, 1H), 3.48-3.53 (m, 1H), 2.62-2.67 (m, 1H), 2.27-2.28 (m, 1H); α-anomer, ¹H NMR (500 MHz, DMSO-d₆) δ 8.38 (s, 1H), 7.81 (br, 2H), 6.24-7.27 (buried in β-1′-H, 1H), 5.50 (d, J=3.9 Hz, 1H), 4.84 (t, J=5.7 Hz, 1H), 4.31-4.30 (m, 1H), 4.10-4.11 (m, 1H), 3.43-3.44 (m, 1H), 3.16 (d, J=5.3 Hz, 1H), 2.70-2.74 (m, 1H), 2.27-2.28 (m, 1H).

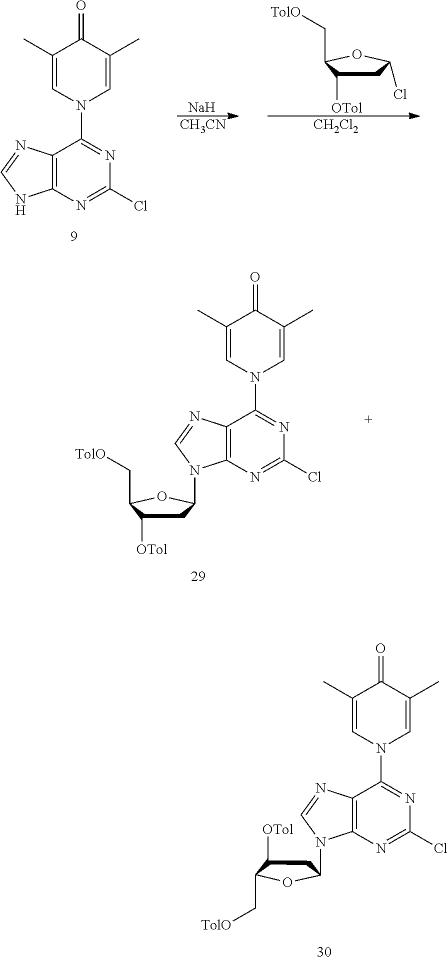

### Glycosylation of 2-chloro-6-(3,5-dimethyl-4-oxopyridin-1-yl)purine

A mixture of 2-chloro-6-(3,5-dimethyl-4-oxopyridin-1-yl)purine (0.69 g, 2.5 mmol) and sodium hydride (60% w/w suspension, 0.12 g, 3.0 mmol) in dried CH₃CN (25 mL) was stirred at ambient temperature under N₂ for 23 h. The solution was chilled to 0° C., and a solution of 2-deoxy-3,5-di-O-(p-toluoyl)-α-D-erythro-pentofuranosyl chloride (1.1 mmol) in cold, dried CH₂Cl₂ (10 mL, C) was added with a syringe. The reaction mixture was then stirred 0° C. for 5 h. The reaction mixture was then concentrated till dryness to give a yellow residue, which was dissolved in DCM (100 mL), washed with saturated aqueous NaHCO₃ (50 mL) and brine (50 mL), and dried from anhydrous MgSO₄. The resulting solution was concentrated to dryness to give a yellow foam (1.56 g), which was analyzed by ¹H NMR as a crude mixture of two anomers (α/β, 1:2.8).

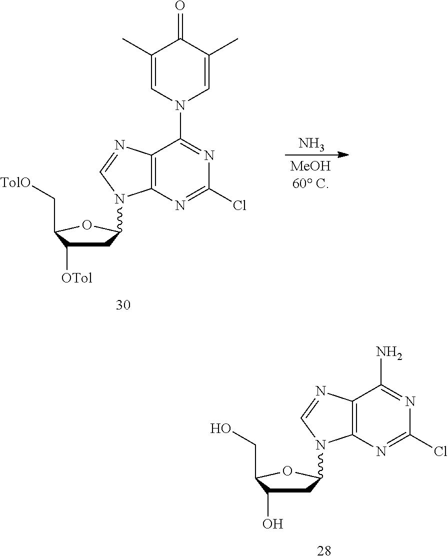

### Aminolysis of 2-chloro-6-(3,5-dimethyl-4-oxopyridin-1-yl)-2′-deoxy-3,5′-di-O-(p-toluoyl)-D-erythro-pentofuranosylpurines

2-Chloro-6-(3,5-dimethyl-4-oxopyridin-1-yl)-[2′-deoxy-3′,5′-di-O-(p-toluoyl)-α/β-D-erythro-pentofuranosyl]purine (1.39 g, crude) in NH₃/MeOH (7 M, 50 mL) in a sealed 100 mL pressure tube was stirred at 60° C. for 48 h, and TLC analysis showed nearly complete reaction. Volatiles were evaporated, and the residue was analyzed by ¹H NMR as a crude mixture of two anomers (α/β, 1:2.8).

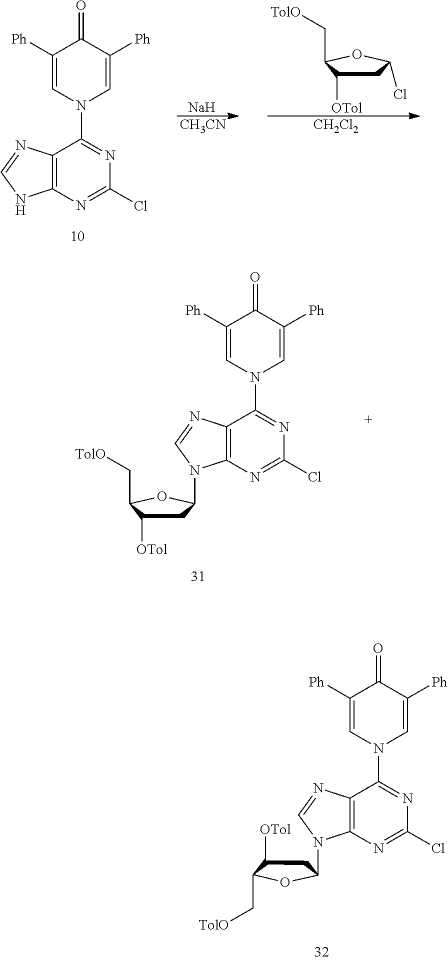

### Glycosylation of 2-chloro-6-(4-oxo-3,5-diphenylpyridin-1-yl)purine

A mixture of 2-chloro-6-(4-oxo-3,5-diphenylpyridin-1-yl)purine (0.72 g, 1.8 mmol) and sodium hydride (60% w/w suspension, 88 mg, 2.2 mmol) in dried CH₃CN (20 mL) was stirred at ambient temperature under N₂ for 21 h. The solution was chilled to 0° C., and a solution of 2-deoxy-3,5-di-O-(p-toluoyl)-α-D-erythro-pentofuranosyl chloride (1.1 mmol) in cold, dried CH₂Cl₂ (10 mL, C) was added with a syringe. The reaction mixture was then stirred 0° C. for 2 h, and allowed to gradually warm to ambient temperature over 2 h. The reaction mixture was then coated onto a limited amount of silica gel and was chromatographed (40 g silica gel, MeOH/CH₂Cl₂, 1:90 to MeOH/CH₂Cl₂, 1:30) to give 2-chloro-6-(4-oxopyrindinyl)-2′-deoxy-3′,5′-di-O-(p-toluoyl)-α/β-D-erythro-pentofuranosylpurine as a mixture of two anomers (1.16 g).

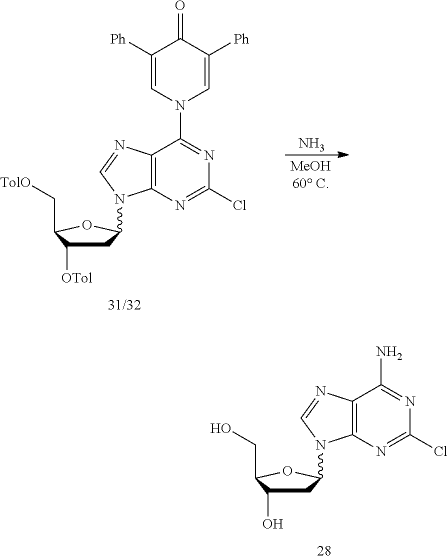

### Aminolysis of 2-chloro-6-oxo-3,5-diphenylpyridin-1-yl)-2′-deoxy-3,5′-di-O-(p-toluoyl)-D-erythro-pentofuranosylpurines

2-Chloro-6-(4-oxo-3,5-diphenylpyridin-1-yl)-[2′-deoxy-3′,5′-di-O-(p-toluoyl)-α/β-D-erythro-pentofuranosyl]purine (0.94 g, crude) in NH₃/MeOH (7 M, 30 mL) in a sealed 100 mL pressure tube was stirred at 60° C. for 42 h, and TLC analysis showed nearly complete reaction. The reaction was stirred at 60° C. for additional 21 h, and TLC showed nearly complete reaction. Volatiles were evaporated, and the residue was dissolved in 1:1 methanol/DCM (60 mL). The insoluble solid was removed by filtration, and the filtrate was concentrated till dryness. The resulting residue was dissolved in water (20 mL), washed with DCM (10 mL×2), and concentrated till dryness. The residue was analyzed by ¹H NMR as a mixture of two anomers (α/β, 1:7.3).

### Preparation and stability of 1-methyl-3-(purin-6-yl)-1H-imidazol-3-ium chloride

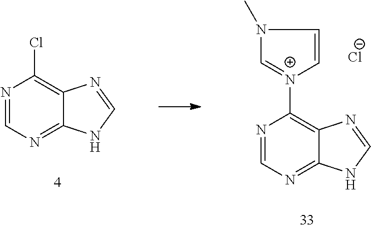

Preparation: 6-chloropurine (0.31 g, 2.0 mmol) in N-methylimidazole (1.0 mL) was stirred under nitrogen at 120° C. for 2 h. Heavy precipitation was observed. The reaction mixture was then cooled to room temperature, and poured into EtOAc (8 mL). The solid was collected and thoroughly washed with DCM (8 mL×2) and dried under vacuum to give 1-methyl-3-(purin-6-yl)-1H-imidazol-3-ium chloride.

Stability: (a) 1-Methyl-3-(purin-6-yl)-1H-imidazol-3-ium chloride (15 mg) was stirred in MeOH (1 mL) at room temperature for 5 h, and analysis of reaction mixture showed starting material (m/z=201 (93%),) and water/methanol adducts (m/z=219 (4%), 251 (2%), 269 (2%)) of imidazole. Displacement product by methanolysis was less than 0.5%. (b) 1-Methyl-3-(purin-6-yl)-1H-imidazol-3-ium chloride (15 mg) was stirred in MeOH (1 mL) at the presence of K₂CO₃ (6 eq.) at room temperature for 5 h, and analysis of reaction mixture showed water/methanol adducts of imidazole (m/z=219 (59%), 251 (26%), 269 (4%)) as major products. Displacement product by methanolysis was about 1%. (c) 1-Methyl-3-(purin-6-yl)-1H-imidazol-3-ium chloride (15 mg) was stirred in MeOH (1 mL) at the presence of KOH (10 eq.) at room temperature for 5 h, and analysis of reaction mixture showed water/methanol adducts of imidazole (m/z=219 (63%), 251 (28%), 269 (5%)) were detected as major products. Displacement product by methanolysis was less than 2%.

The formation of water adducts can be minimized or eliminated by using 2-substituted imidazoles such as 2-alkylimidazoles.

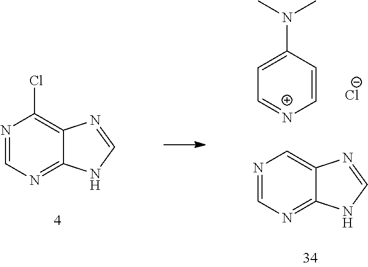

### Preparation and stability of 4-(dimethylamino)-1-(9H-purin-6-yl)pyridin-1-ium chloride

A mixture of 4-N,N-dimethylaminopyridine (21.4 mmol) and 6-chloropurine (2.98 g, 19.4 mmol) in DMF (30 mL) was stirred at room temperature under nitrogen for 18 h. The reaction temperature was then elevated to 135° C., and was stirred at that temperature for another 5.5 h. The reaction mixture was cooled, and poured into ethyl acetate (100 mL), and the solid was collected by filtration. This solid was further washed with limited amount of DMF and then ethyl acetate (30 mL) and dried under vacuum to give 6-(4-N,N-dimethyl aminopyridin-1-yl)purine chloride. Recrystallization from water gave a tainted white solid.

Stability: 4-(dimethylamino)-1-(9H-purin-6-yl)pyridin-1-ium chloride (5 mg) was stirred in MeOH (0.5 mL) at room temperature for 4 days; no detectable changes were observed by either TLC or ¹H NMR analysis. In a parallel experiment, to the methanolic solution of 4-(dimethylamino)-1-(9H-purin-6-yl)pyridin-1-ium chloride were added the K₂CO₃ (5 mg) and water (0.02 mL), and the mixture was stirred for 72 h, then KOH (5 mg) was added, and the mixture was stirred for additional 24 h. Volatiles were evaporated, and the residue was analyzed by ¹H NMR. No displacement of DMAP either by water or methanol was detected in spite of suspected decomposition of this substituted purine under strong basic conditions.

### Preparation of 1-(2-chloro-9H-purin-6-yl)-4-(dimethylamino)pyridin-1-ium chloride

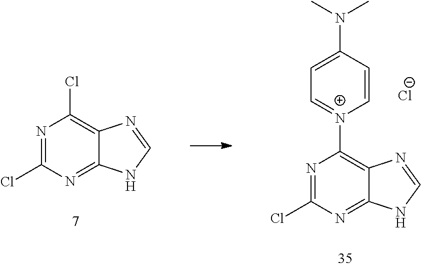 [SMILES: Clc1nc(Cl)c2nc[nH]c2n1 ; CN(C)c1cc[n+](-c2nc(Cl)nc3[nH]cnc23)cc1 ; [Cl-]]

A mixture of 2,6-dichloropurine (2.0 g, 10.6 mmol) and 4-N,N-dimethylamiopyridine (31.8 mmol) in DMF (36 mL) is stirred under nitrogen at 65° C. for 23 h. The reaction mixture is cooled to rt, and poured into ethyl acetate (100 mL). The precipitated solid is collected by filtration, washed with limited amount of DMF, and then ethyl acetate, and dried under vacuum to give the title compound.

### Preparation of 1-(2-amino-9H-purin-6-yl)-4-(dimethylamino)pyridin-1-ium chloride

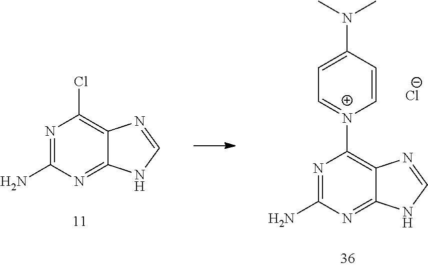 [SMILES: Nc1nc(Cl)c2nc[nH]c2n1 ; CN(C)c1cc[n+](-c2nc(N)nc3[nH]cnc23)cc1 ; [Cl-]]

A mixture of 2-amino-6-chloropurine (2.0 g, 11.8 mmol) and 4-N,N-dimethylaminopyridine (71.8 mmol) in DMF (36 mL) is stirred under nitrogen at 65° C. for 20 h. The mixture is then stirred at 130° C. for 3 days, and then cooled to room temperature and poured in to ethyl acetate (100 mL). The solid is collected by filtration, washed with limited amount of DMF, and then ethyl acetate, and dried under vacuum to give the title compound.

### Preparation of 9-ethyl-6-methoxypurine

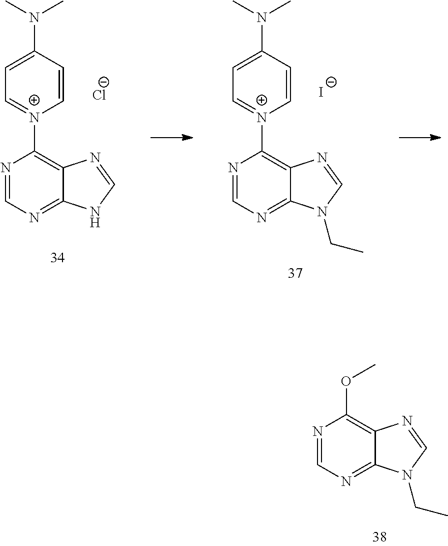 [SMILES: CN(C)c1cc[n+](-c2ncnc3[nH]cnc23)cc1 ; [Cl-] ; CCn1cnc2c(-[n+]3ccc(N(C)C)cc3)ncnc21 ; [I-] ; CCn1cnc2c(OC)ncnc21]

Method a: A mixture of 4-(dimethylamino)-1-(9H-purin-6-yl)pyridin-1-ium chloride (0.55 g, 2 mmol) and sodium hydride (60% w/w suspension, 0.09 g, 2.2 mmol) in anhydrous DMF (10 mL) was stirred at ambient temperature under N2 for 40 min. Ethyl iodide (0.94 g, 0.48 mL, 6 mmol) was then added dropwise with a syringe to the resulting 6-(4-(dimethylamino)pyridin-1-ium-1-yl)purin-9-ide. The reaction mixture was then stirred at ambient temperature for 18 h. Methanol (1 mL) and KOH (0.12 g, 2.1 mmol) were added, and the resulting mixture was stirred at room temperature for 15 min. Analysis of the reaction mixture showed no detectable N-7 alkylated product. The reaction mixture was then diluted in saturated ammonium chloride aqueous solution (18 mL), and extracted with DCM (18 mL×2). The extracts were combined and washed with brine (18 mL), and dried from anhydrous MgSO₄. Volatiles were evaporated in vacuo, and the residue was coated onto silica gel and separated by column chromatograph (silica gel, CH₂Cl₂ then MeOH/CH₂Cl₂) to give the title compound: ¹H NMR (500 MHz, DMSO-d₆) δ 8.52 (s, 2H), 8.40 (s, 1H), 4.08 (s, 3H), 4.26 (q, J=7.3 Hz, 2H), 1.42 (t, J=7.3 Hz, 3H).

It was observed that displacement of 6-(4-(dimethylamino)pyridin-1-ium-1-yl) by methanolysis of the alkylated product was about 80% complete within 2 min by addition of methanol to the reaction mixture (including K₂CO₃).

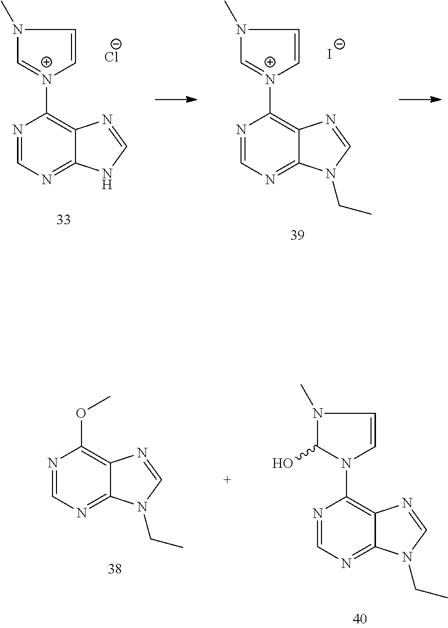 [SMILES: Cn1cc[n+](-c2ncnc3[nH]cnc23)c1 ; [Cl-] ; CCn1cnc2c(-[n+]3ccn(C)c3)ncnc21 ; [I-] ; CCn1cnc2c(OC)ncnc21 ; CCn1cnc2c(N3C=CN(C)C3O)ncnc21]

Method b: A mixture of 1-methyl-3-(purin-6-yl)-1H-imidazol-3-ium chloride (0.24 g, 1 mmol), ethyl iodide (0.47 g, 0.24 mL, 3.0 mmol) and K₂CO₃ (0.41 g, 3.0 mmol) in anhydrous DMF (3 mL) was stirred at ambient temperature under N₂ for 20 h. Methanol (1 mL) and KOH (0.12 g, 2.1 mmol) were added, and the resulting mixture was stirred at room temperature for 15 min. Analysis of the reaction mixture showed no detectable N-7 alkylated product. The reaction mixture was then diluted in saturated ammonium chloride aqueous solution (18 mL), and extracted with DCM (18 mL×2). The extracts were combined and washed with brine (18 mL), and dried from anhydrous MgSO₄. Volatiles were evaporated in vacuo, and the residue was coated onto silica gel and separated by column chromatograph (silica gel, CH₂Cl₂ then MeOH/CH₂Cl₂) to give the title compound. Also isolated was 1-(9-ethyl-9H-purin-6-yl)-3-methyl-2,3-dihydro-1H-imidazol-2-ol.

It was observed that displacement of 6-(3-methylimidazol-1-ium-1-yl) by methanolysis of the alkylated product was about 85% complete within 2 min by addition of methanol to the reaction mixture (including K₂CO₃). Also formed were H₂O/methanol adducts as major products.

### Preparation of 2-chloro-9-ethyl-6-methoxypurine

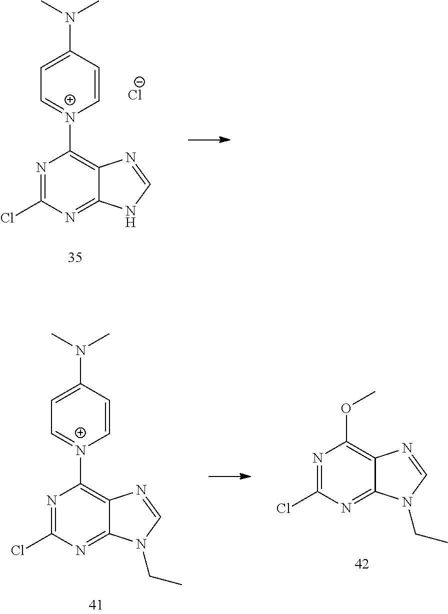 [SMILES: CN(C)c1cc[n+](-c2nc(Cl)nc3[nH]cnc23)cc1 ; [Cl-] ; CCn1cnc2c(-[n+]3ccc(N(C)C)cc3)nc(Cl)nc21 ; CCn1cnc2c(OC)nc(Cl)nc21]

A mixture of 1-(2-chloro-9H-purin-6-yl)-4-(dimethylamino)pyridin-1-ium chloride (0.25 g, 0.80 mmol), ethyl iodide (0.37 g, 0.19 mL, 2.40 mmol) and K₂CO₃ (0.33 g, 2.40 mmol) in anhydrous DMF (5 mL) is stirred at ambient temperature under N₂ for 20 h. TLC analysis shows nearly complete reaction. Methanol (1 mL) and KOH (0.12 g, 2.1 mmol) are added, and the resulting mixture is stirred at room temperature for 1 h. The reaction mixture is then diluted in saturated ammonium chloride aqueous solution (18 mL), and extracted with DCM (18 mL×2). The extracts are combined and washed with brine (18 mL), and dried from anhydrous MgSO₄. Volatiles of filtrate are evaporated in vacuo and the residue is chromatographed (silica gel, CH₂Cl₂ then MeOH/CH₂Cl₂) to give the title compound.

### Preparation of 2-amino-9-ethyl-6-methoxypurine

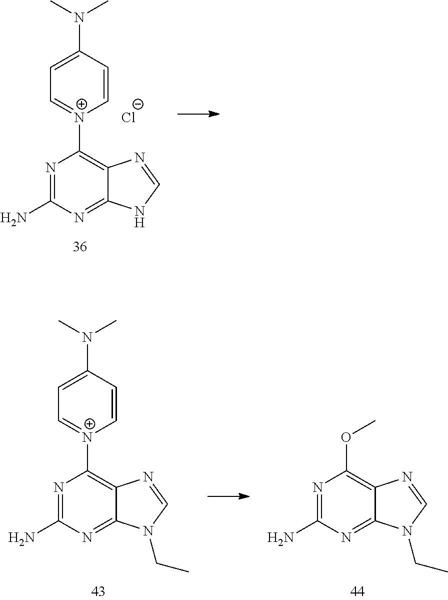 [SMILES: CN(C)c1cc[n+](-c2nc(N)nc3[nH]cnc23)cc1 ; [Cl-] ; CCn1cnc2c(-[n+]3ccc(N(C)C)cc3)nc(N)nc21 ; CCn1cnc2c(OC)nc(N)nc21]

A mixture of 1-(2-amino-9H-purin-6-yl)-4-(dimethylamino)pyridin-1-ium chloride (0.58 g, 2.0 mmol), ethyl iodide (0.47 g, 0.24 mL, 3.0 mmol) and K₂CO₃ (0.41 g, 3.0 mmol) in anhydrous DMF (10 mL) is stirred at ambient temperature under N2 for 16 h. The reaction temperature is elevated to 65° C., and the reaction mixture stirred at 65° C. for 24 h. Methanol (1 mL) and KOH (0.12 g, 2.1 mmol) are added, and the resulting mixture is stirred at room temperature for 1 h. The reaction mixture is then diluted in saturated ammonium chloride aqueous solution (20 mL), and extracted with DCM (20 mL×2). The extracts are combined and washed with brine (20 mL), and dried from anhydrous MgSO₄. Volatiles of filtrate are evaporated in vacuo and the residue is chromatographed (20 g silica gel, CH₂Cl₂ then MeOH/CH₂Cl₂) to give the title compound.

### Glycosylation of 1-(2-chloro-9H-purin-6-yl)-4-(dimethylamino)pyridin-1-ium chloride

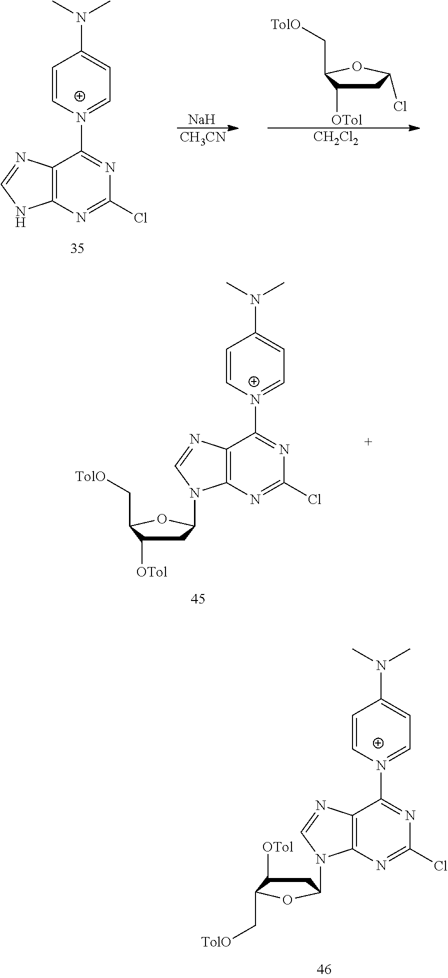

A mixture of 1-(2-chloro-9H-purin-6-yl)-4-(dimethylamino)pyridin-1-ium chloride (1 mmol) and sodium hydride (60% w/w suspension, 1.2 mmol) in dried CH₃CN (10 mL) is stirred at ambient temperature under N₂ for 1 h. The solution is chilled to 0° C., and a solution of 2-deoxy-3,5-di-O-(p-toluoyl)-α-D-erythro-pentofuranosyl chloride (1.1 mmol) in cold, dried CH₂Cl₂ (10 mL, C) is added with a syringe. The reaction mixture is then stirred at 0° C. for 2 h, and allowed to gradually warm to ambient temperature over 2 h. Suspension is removed by filtration, and filtrate is concentrated till dryness to give a yellow residue, which is dissolved in DCM (20 mL) and the insoluble solid of small amount is removed by filtration. The filtrate is concentrated to give a slight yellow residue.

### Aminolysis of 2-chloro-6-(4-N,N-dimethylaminopyridin-1-yl)-2′-deoxy-3′,5′-di-O-(p-toluoyl)-D-erythro-pentofuranosylpurine chlorides

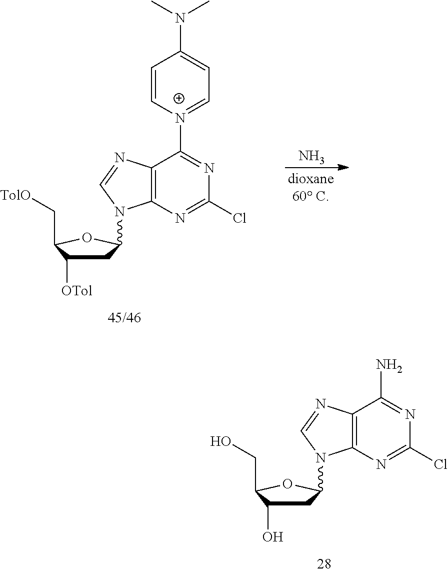

2-Chloro-6-(4-N,N-dimethylaminopyridin-1-yl)-[2′-deoxy-3′,5′-di-O-(p-toluoyl)-α/β-D-erythro-pentofuranosyl]purine chlorides (1.0 g, crude) in NH₃/dioxane (0.5 M, 30 mL) in a sealed 100 mL pressure tube is stirred at 60° C. Volatiles are evaporated, and the residue is dissolved in 1:1 methanol/DCM, and coated onto limited amount of silica gel. Column chromatography (silica gel, MeOH/DCM) gives a mixture of two anomers.

### Preparation of guanosine 5′-triphosphate

Step a: Polymer supported DMAP (˜3.0 mmol/g, 1 g) and 2-isobutyramido-6-chloropurine (1.44 g, 6 mmol) in anhydrous DMF (30 mL) are shaken at 120° C. for 16 h in a sealed tube, and then the mixture is allowed to cool to room temperature. The solid is collected by filtration, washed with DMF (20 mL×3), THF (20 mL), and DCE (20 mL), sequentially, and dried under vacuum for 10 min.

Small sample of this solid is shaked with EtI (in excess) in DMF overnight at the presence of K₂CO₃, and is cleaved by treatment with 1 M sodium methoxide solution in methanol at room temperature for 15 min, and the reaction yield is calculated based on weight loss and the amount of 9-ethyl-2-isobutylamido-6-methoxypurine.

Step b (1): N,O-Bis(trimethylsilyl)acetamide (BSA) (1.56 mL, 1.26 g, 6.0 mmol) is added to a suspension of the solid from step a in dried DCE (20 mL). The mixture is shaken at 80° C. for 30 min, and then is allowed to cool to room temperature. The solid is collected by filtration, washed with DCE (20 mL) and dried toluene (20 mL), and dried under vacuum for 15 min. The resulting solid is then suspended in dried toluene (20 mL), and 2,3-O-diacetyl-5-O-tert-butyldiphenylsilylribofuranosyl acetate (1.85 g, 3.6 mmol) and TMSOTf (0.78 mL, 1.86 g, 4.8 mmol) are added. The mixture is shaken for 2 h at 80° C., and then is allowed to cool to room temperature. The solid is collected by filtration, washed with DCE (20 mL×3) and THF (20 mL), sequentially, and dried under vacuum for 10 min.

or

Step b (2): To a suspension of the solid from step a in dried CH₃CN (20 mL) are added 2,3-O-diacetyl-5-O-tert-butyldiphenylsilylribofuranosyl acetate (1.85 g, 3.6 mmol) and SnCl₄ (0.60 mL, 1.32 g, 5.1 mmol), and the mixture is shaken at ambient temperature for 4 h. The solid is collected by filtration, washed with dried CH₃CN (20 mL×3), and THF (20 mL) sequentially, and dried under vacuum for 10 min.

Step c: The solid from step b (1) or b (2) is suspended in 1M TBAF in THF (20 mL), and shaken at ambient temperature for 16 h. The solid is collected by filtration, washed with THF (20 mL), DMF (20 mL×3), and anhydrous pyridine (20 mL), sequentially, and dried under vacuum for 10 min.

Step d: The solid from step c is suspended in anhydrous DMF/pyridine (1:1, 10 mL), and 2-chloro-4H-benzo[d][1,3,2]dioxaphosphinin-4-one (0.67 g, 3.3 mmol) in dioxane (3.3 mL) is added. The mixture is shaken for 15 min. 0.84 M Tributylammonium pyrophosphate in DMF (3.9 mL, 3.3 mmol) and tributylamine (2.7 mL, 11.4 mmol) are added. The mixture is shaken at ambient temperature for 0.5 h, and then cooled to 0° C. Precooled iodine solution in pyridine-water (98:2) (0.3 M, 10.5 mL) is added, and the mixture is shaken at ambient temperature for 15 min. The solid is collected by filtration, washed with DMF (20 mL), THF (20 mL), and acetone (20 mL), sequentially, and dried under vacuum for 10 min.

Step e: The solid from step d is suspended in 1 M lithium hydroxide in H₂O (20 mL), and the resulting mixture is shaken for 16 h. The solid is removed by filtration, and the filtrate is concentrated by lyophilization to give a solid, which is separated by a DEAE Sephadex column eluted with gradient TEAB buffer (pH 7.5, 0.05-0.8 M). The fractions of the product are combined and concentrated to dryness by lyophilization to give the title compound.

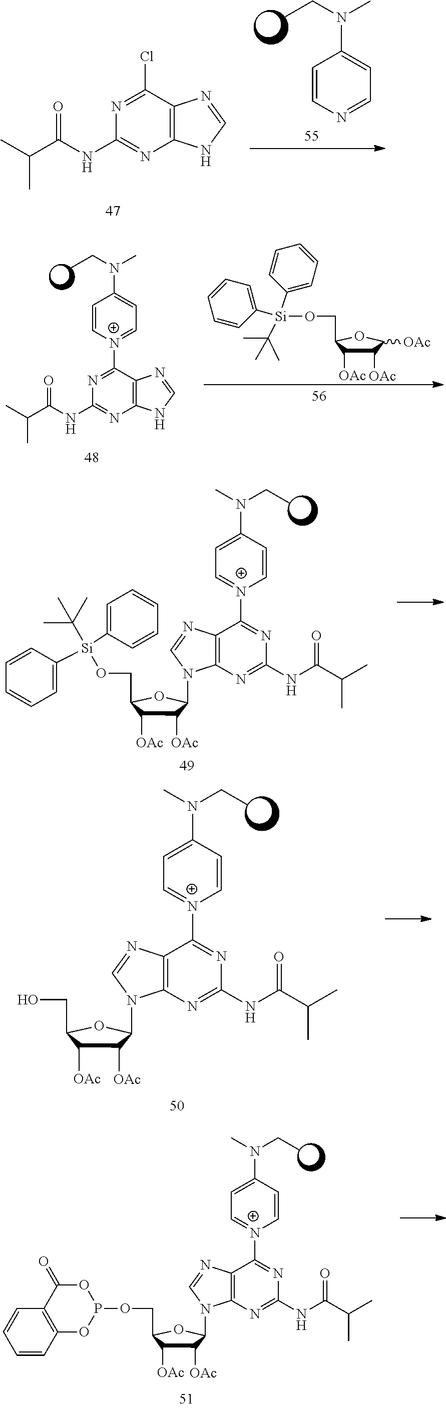

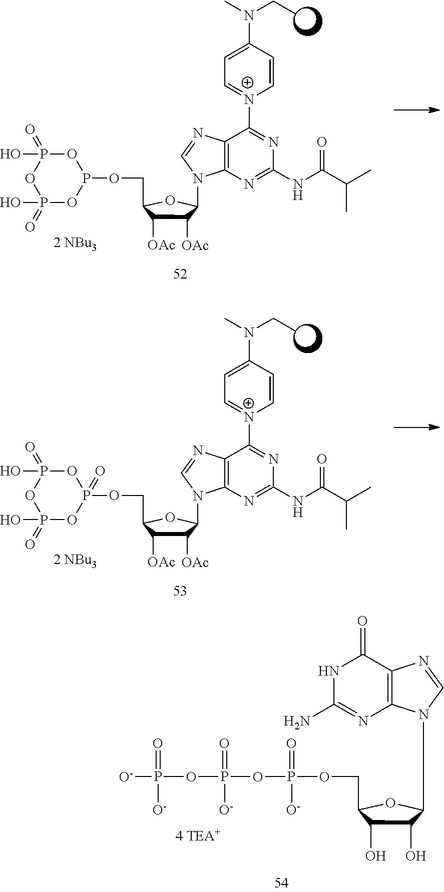

### Preparation of 2′-deoxyguanosine 5′-triphosphate

Step a: Polymer supported DMAP (˜3.0 mmol/g, 1 g) and 2′-deoxy-2-trifluoroacetyl-6-pentafluorophenylguanosine (1.59 g, 3 mmol) in anhydrous DMF (10 mL) are shaken at 80° C. for 16 h, and then is allowed to cool to room temperature. The solid is collected by filtration, washed with DMF (20 mL×3), THF (20 mL), and acetone (20 mL), sequentially, and dried under vacuum for 10 min.

Small sample of this solid is cleaved by treatment with 1 M sodium methoxide solution in methanol at room temperature, and the reaction yield is calculated based on weight loss, and the amount of 2′-deoxy-6-methoxyguanosine.

Step b: The solid from step a is suspended in trimethyl phosphate (20 mL). The mixture is then cooled to −15° C. and dry proton sponge is added (3 equiv.). The mixture is shaken for 20 min. Phosphoryl oxychloride is added (1.2 equiv.). The mixture is shaken for 2 h at −15° C., and then a mixture of Bu₃N (4 equiv.) and 1 M H₂P₂O₇ (Bu₃NH)₂ in DMF (4 equiv.) is added. The mixture is shaken for additional 2 h at −15° C. The solid is collected by filtration, washed with DMF (20 mL×3) and THF (20 mL), sequentially, and dried under vacuum for 10 min.

Step c: The solid from step b is suspended in 1 M lithium hydroxide in H₂O (20 mL), and the resulting mixture is shaken for 16 h. The solid is removed by filtration, and the filtrate is concentrated by lyophilization to give a solid, which is separated by a DEAE Sephadex column eluted with TEAB buffer (pH 7.5, 0.05-0.8 M). The fractions of the product are combined and concentrated to dryness by lyophilization to give the title compound.

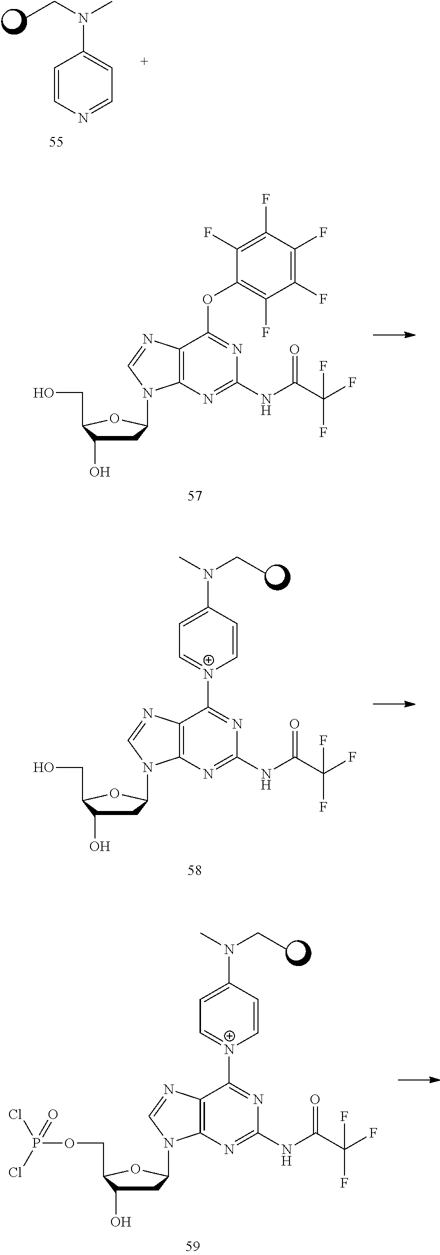

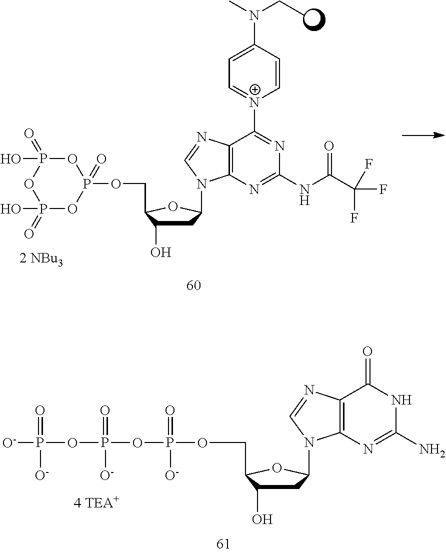

What is claimed is:

1. A method for preparing an N-9 purine nucleoside comprising:
(a) contacting a substituted purine of Formula I 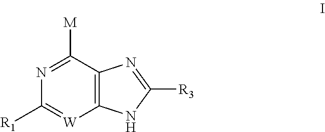
with a glycosylating agent or an alkylating agent in the presence of a base, or Lewis acid, or catalyst such as Pd(PPh₃), or under Mitsunobu reaction condition, where W is selected from —N—, —CH— and CR₂, and M is a substituted or unsubstituted pyridinium, a substituted imidazolium, or 4-oxopyridin-1-yl: 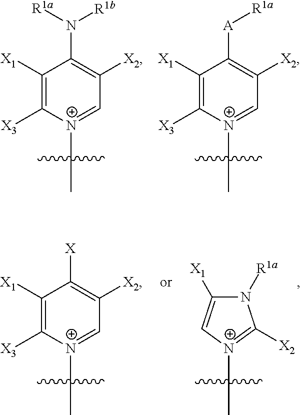
and where R₁, R₂, R₃, R^{1a}, R^{1b}, X, X₁, X₂, and X₃ are selected as follows: (i) each R₁, R₂, R₃, X, X₁, X₂, and X₃ is independently selected from hydrogen, C₁₋₁₀ alkyl, C₁₋₁₀ alkoxy, C₁₋₁₀ alkylthio, halogen, amino, hydroxyl, C₁₋₁₀ alkylamino, di-C₁₋₁₀ alkylamino, C₁₋₁₀ acylamino, trialkylsilyl, aryl, and heteroaryl, preferably with X₃ selected as hydrogen; (ii) R^{1a} and R^{1b} is independently selected from C₁₋₁₀ alkyl, aryl, and heteroaryl; (iii) R^{1a}, R^{1b}, X₁, X₂ together form cyclic rings represented by, but not limited to, the following structures: 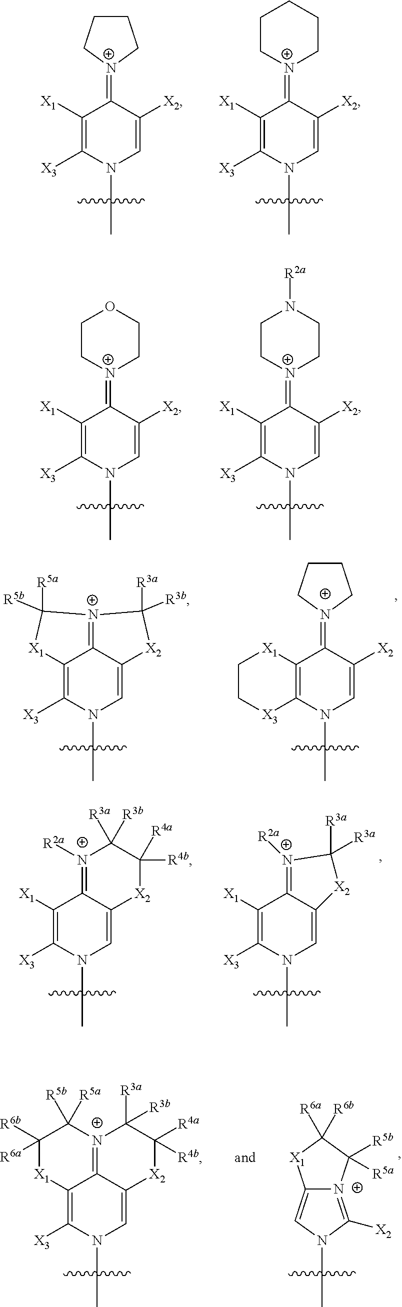
where R^{2a-6a}, and R^{3b-6b} are independently selected from hydrogen, C₁₋₁₀ alkyl, C₁₋₁₀ alkoxy, C₁₋₁₀ alkylthio, halogen, amino, C₁₋₁₀ alkylamino, di-C₁₋₁₀ alkylamino, C₁₋₁₀ acylamino, trialkylsilyl, aryl, and heteroaryl, preferably with X₃ selected as hydrogen; (iv) R^{1a}, R^{1b}, X1, or X2 is bound to an inorganic and/or organic particulate solid support as represented by following examples: 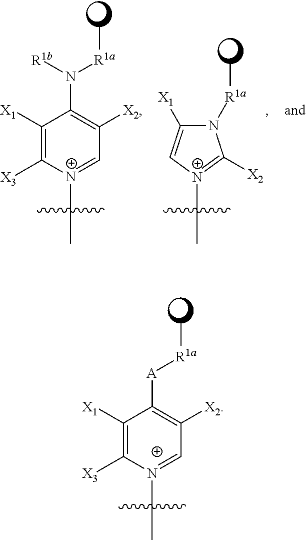
Non-limiting examples of solid supports include silica gel, silicates, alumina, glass beads, polystyrenes, polyacrylates, and other organic resins; and where A is O, S, or alkyl.
(b) contacting the 6-substituted purine nucleoside from step (a) with a nucleophile to obtain a nucleoside derivative of Formula II 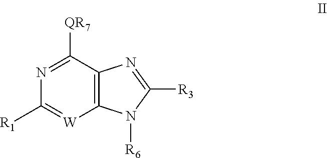
where R₆ is a glycosyl or alkyl group, and Q is independently selected from O, NH, S, and Se, and where R₇ is hydrogen, alkyl, aryl, or heteroaryl.
(c) global deprotection of the nucleoside derivative from step (b) provides a purine nucleoside, and this step may not be needed if concomitant deprotection and nucleophilic displacement happen in step (b).

2. The method of claim 1, wherein W is —N—, R₁ is Cl, and R₃ is H, and wherein the glycosylating agent is an activated and hydroxyl-protected 2-deoxy-α-D-erythro-pentofuranosyl compound in step (a), and the displacing nucleophile is ammonia, to obtain (2R,3S,5R)-5-(6-amino-2-chloro-9H-purin-9-yl)-2-(hydroxymethyl)tetrahydrofuran-3-ol (2-chloro-2′-deoxyadenosine, 2-CdA, Cladribine).

3. The method of claim 1, wherein W is —N—, R₁ is Cl, and R₃ is H, and wherein the glycosylating agent is an activated and hydroxyl-protected 2-deoxy-2-fluoro-α-D- or L-arabinofuranosyl compound in step (a), and the displacing nucleophile is ammonia, to obtain (2R,3R,4S,5R)-5-(6-amino-2-chloro-9H-purin-9-yl)-4-fluoro-2-(hydroxymethyl)tetrahydrofuran-3-ol (Clofarabine).

4. The method of claim 1, wherein W is —N—, R₁ is F, and R₃ is H, and wherein the glycosylating agent is an activated and hydroxyl-protected α-D-arabinofuranosyl compound in step (a), and the displacing nucleophile is ammonia, to obtain (2R,3S,4S,5R)-2-(6-amino-2-fluoro-9H-purin-9-yl)-5-(hydroxymethyl)tetrahydrofuran-3,4-diol (Fludarabine).

5. The method of claim 1, wherein W is —N—, R₁ is amino or protected amino, and R₃ is H, and the displacing nucleophile is ammonia, to obtain ((2R,4R)-4-(2,6-diamino-9H-purin-9-yl)-1,3-dioxolan-2-yl)methanol (Amdoxovir, DAPD), or its precursors, or its prodrugs.

6. The method of claim 1, wherein W is —N—, R₁ is amino or protected amino, and R₃ is H, and wherein the alkylating reagent is an activated and hydroxyl-protected cyclopentane compound such as its epoxide, sulfonates, and halides, or allylic triflate, acetate, and benzoate, or a cyclopentanol in step (a), and the displacing nucleophile is hydroxide (hydrolysis), to obtain 2-amino-9-((1S,3R,4S)-4-hydroxy-3-(hydroxymethyl)-2-methylenecyclopentyl)-1H-purin-6(9H)-one (Entecavir).

7. The method of claim 1, wherein W is —N—, R₁ is amino or protected amino, and R₃ is H, and the displacing nucleophile is hydroxide (hydrolysis), to obtain 2-amino-9-((1R,2R,3S)-2,3-bis(hydroxymethyl)cyclobutyl)-1H-purin-6(9H)-one (Lobucavir), Acyclovir, Ganciclovir, Penciclovir (PCV), or their precursors, or their prodrugs.

8. The method of claim 1, wherein W is —N—, R₁ is amino or protected amino, and R₃ is H, the 6-heteroarylium is displaced by a hydrogen to obtain Famciclovir (FCV), or their prodrugs.

9. The method of claim 1, wherein W is —N—, R₁ is hydrogen, and R₃ is H, and the 6-heteroarylium is displaced by ammonia, or by azide ion followed by further transformation such as Staudinger reaction to give Adefovir, Tenofovir, or their precursors, or their prodrugs.

10. The method of claim 1, wherein W is —N—, R₁ is hydrogen, and R₃ is H, and the 6-heteroarylium is displaced by hydrolysis to give 2′,3′-dideoxyinosine (DDI) or its precursors.

11. The method of claim 1, wherein W is —N—, R₁ is amino or protected amino, and R₃ is H, and wherein the alkylating reagent is an activated and hydroxyl-protected cyclopentene compound or it precursors in step (a), and the displacing nucleophile is cyclopropylamine, to obtain ((1S,4R)-4-(2-amino-6-(cyclopropylamino)-9H-purin-9-yl)cyclopent-2-en-1-yl)methanol (Abacavir).

12. The method of claim 1, wherein W is —N—, R₁ is amino or protected amino, and R₃ is H, and wherein the 6-heteroarylium is displaced by methanolysis to give (2R,3S,4S,5R)-2-(2-amino-6-methoxy-9H-purin-9-yl)-5-(hydroxymethyl)tetrahydrofuran-3,4-diol (Nelarabine) or its precursors, or its prodrugs.

13. The method of claim 1, wherein R^{1a}, R^{1b}, X, X₁, X₂ or X₃ is bound to an inorganic and/or organic particulate solid support for preparing purine nucleoside libraries on solid supports comprises:
(a) contacting a compound having Formula VII 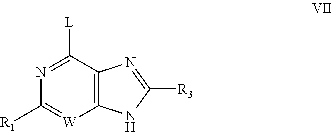
with solid supported pyridine or imidazole in certain solvent to form 6-heteroarylpurine having formula XXXX 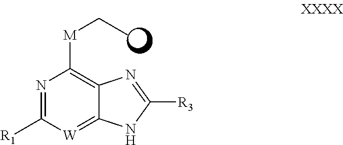
(b) contacting the solid supported 6-heteroarylpurine with a base in a polar solvent followed by contacting an activated and hydroxyl-protected alkylating reagent or a glycosylating reagent in a less polar solvent to form a 6-heteroarylpurine nucleoside product having formula XXXXI 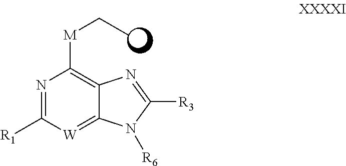
(c) contacting the 6-heteroarylium purine nucleoside product from step (b) with a nucleophile in a third solvent to displace solid supported heteroaryl (M), followed by further chemical steps as needed, to obtain a purine nucleoside. Alternatively, these chemical steps can precede the displacement step for easy purification.

14. A method for preparing purine nucleoside triphosphates, monophosphates, diphosphates, or ProTides on solid supports comprises:
(a) formation of a solid supported 6-heteroarylpurine nucleoside product having formula XXXXII after partial or full deprotection of glycosyl moiety; 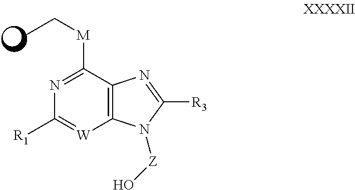
(b) phosphorylation of the 6-heteroarylpurine nucleoside product from step (a) to form a 6-heteroarylium purine nucleotide triphosphate having formula XXXXIII, or a monophosphate, a diphosphate, or a ProTide; 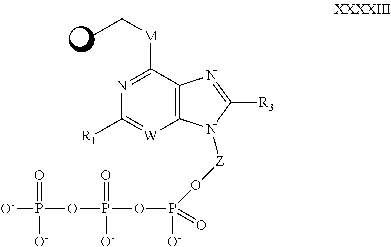
(c) contacting the 6-heteroarylium purine nucleotide product from step (b) with a nucleophile in a third solvent to displace solid supported 6-heteroarylium, followed by further chemical steps as needed to provide a purine nucleotide triphosphate having formula XXXXIV, or a monophosphate, a diphosphate, or a ProTide. Alternatively, these chemical steps can precede the displacement step for easy purification; 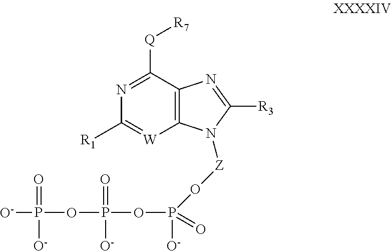
wherein Z is a moiety derivable by removal of 5′-hydroxyl radical from a glycosylated sugar, a sugar derivative, or its analogues.

15. A compound of Formula I 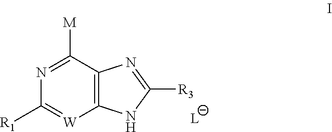
where W is selected from —N—, —CH— and CR₂; and M is a substituted pyridinium or a substituted imidazolium: 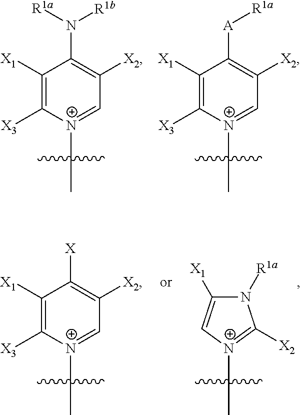
where R₁, R₂, R₃, R^{1a} and R^{1b}, X, X₁, X₂, and X₃ are selected as follows: (i) each R₁, R₂, R₃, X, X₁, X₂, and X₃ is independently selected from hydrogen, C₁₋₁₀ alkyl, C₁₋₁₀ alkoxy, C₁₋₁₀ alkylthio, halogen, amino, hydroxyl, C₁₋₁₀ alkylamino, di-C₁₋₁₀ alkylamino, C₁₋₁₀ acylamino, trialkylsilyl, aryl, and heteroaryl, preferably with X₃ selected as hydrogen; (ii) R^{1a} and R^{1b} is independently selected from C₁₋₁₀ alkyl, aryl, and heteroaryl; (iii) R^{1a}, R^{1b}, X₁, X₂ together form cyclic rings; preferably with X₃ selected as hydrogen; (iv) R^{1a}, R^{1b}, X, X₁, X₂ or X₃ is bound to an inorganic and/or organic particulate solid support;
where A is O, S, or alkyl;
where when substituted 6-pyridyl is 6-(4-N,N-dimethylpyridin-1-yl) or unsubstituted 6-pyridyl, and when R₃ is H, and W is N, R₁ is not Cl or NH₂;
where when X is OH, W is N, and both R₁ and R₃ are hydrogen, then at least one of X₁, X₂, and X₃ is not hydrogen;
where when X is OH, W is N, and both R₁ and R₃ are hydrogen, then either both of X₁ and X₂ are sulfonamides or both are not sulfonamides;
where when X is H, W is N, R₃ is hydrogen, and R₁ is Cl or NH₂, then either both of X₁ and X₂ are substituted carbonyls, or both are not substituted carbonyls, or either of them a carbonyl substituted with functions excluding methyl, ethyl, O-methyl, O-ethyl, NH₂, N-dimethyl, and N-diethyl;
and where L is selected from halides, sulfates, and non-reactive anions;
and pharmaceutically acceptable salts of these compounds.

16. The compounds of claim 15, where W is N, and R₃ is H; and pharmaceutically acceptable salts of these compounds.

17. Mesomeric betaines of compounds of claim 15 of Formula XXXIX; and pharmaceutically acceptable salts of these compounds.

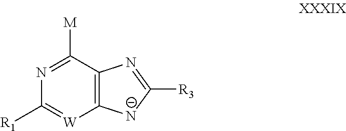

18. Mesomeric betaines of compounds of claim 15, where W is N, and R₃ is H; and pharmaceutically acceptable salts of these compounds.
